(19) United States
(12) Patent Application Publication
(10) Pub. No.: US 2015/0073021 A1
(43) Pub. Date: Mar. 12, 2015

(54) CHEMICALLY MODIFIED CURCUMINS AS INHIBITORS OF ANTHRAX LETHAL FACTOR

(71) Applicants: Anthony ANTONELLI, Syosset, NY (US); Sanford R. SIMON, Stony Brook, NY (US); Yu ZHANG, Port Jefferson Station, NY (US); Lorne M. GOLUB, Smithtown, NY (US); Francis JOHNSON, Setauket, NY (US)

(72) Inventors: Anthony ANTONELLI, Syosset, NY (US); Sanford R. SIMON, Stony Brook, NY (US); Yu ZHANG, Port Jefferson Station, NY (US); Lorne M. GOLUB, Smithtown, NY (US); Francis JOHNSON, Setauket, NY (US)

(73) Assignees: The Research Foundation for The State University of New York, Albany, NY (US); Chem-Master International, Inc., East Setauket, NY (US)

(21) Appl. No.: 14/478,886

(22) Filed: Sep. 5, 2014

Related U.S. Application Data
(60) Provisional application No. 61/874,767, filed on Sep. 6, 2013.

(51) Int. Cl. C07C 225/22; C07C 69/738; C07D 213/50; A61K 31/444; A61K 31/136; A61K 31/216; A61K 31/166; C07C 235/74; A61K 45/06

(52) U.S. Cl. CPC C07C 225/22; C07C 235/74; C07C 69/738; C07D 213/50; A61K 45/06; A61K 31/136; A61K 31/216; A61K 31/166; A61K 31/444
USPC 514/332; 514/646; 514/539; 514/619; 514/621; 514/545; 435/184

(57) ABSTRACT

The present invention provides a method of inhibiting the binding of anthrax lethal factor with protective antigen comprising contacting the anthrax lethal factor with a compound having the structure:

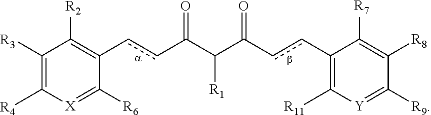

This application claims the benefit of U.S. Provisional Application No. 61/874,767, filed Sep. 6, 2013, the contents of which are hereby incorporated by reference in its entirety.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### BACKGROUND OF THE INVENTION

Bacillus anthracis is the bacterium responsible for the disease anthrax in humans and other animals, and is most often encountered in nature in the form of spores that are highly resistant to environmental damage. Though cutaneous or gastrointestinal anthrax infection can be fatal if not treated promptly, infection is most deadly when these spores are inhaled, as they quickly take up residence in the lymph nodes and begin to destroy or inactivate the local population of macrophages and dendritic cells, a process which is thought to depress the initial immune response and increase the virulence of infection (Mock, M. et al. 2003; Rosenberger, C. M. et al. 2003; Brittingham, K. C. et al. 2005). A major contributing factor to the morbidity of anthrax infection is that it is often asymptomatic until the bacteria have left the lymph nodes and spread to the blood, at which point they begin to multiply at a rate that is very difficult to overcome by antibiotics alone (Dixon, T. C. et al. 1999; Tonello, F. et al. 2009). Especially after the terrorist events of 2001, the threat of anthrax as a biological weapon has made development of more effective treatments for the infection a top priority, though only limited progress has been made over this time.

The primary toxic effects of Bacillus anthracis are caused by secretion of three proteins acting synergistically: lethal factor (LF), the metalloproteinase component; edema factor (EF), a Ca²⁺/calmodulin-dependent adenylate cyclase; and protective antigen (PA), a carrier protein for the former two proteins that binds cell surface receptors and polymerizes to form a pore (Rosenberger, C. M. et al. 2003; Mastrolorenzo, A. et al. 2009). In isolation, LF and EF are benign to cells: they require binding to PA before they can enter cells and exert their cytotoxicity. The complexes with PA are referred to as lethal toxin (LeTx) and edema toxin (ETx), respectively (Mock, M. et al. 2001). Ultimately, evidence shows that the biological activity of LF is the main, and direct cause of anthrax toxicity.

Once secreted, protective antigen molecules bind to a class of cell surface receptors known as anthrax toxin receptors, which allows a furin-like protease on the cell surface to release a 20 kDa portion of the PA amino terminus, leaving the activated form of PA behind in the plasma membrane. Once activated, PA molecules come together to form a heptamer, and are then capable of binding both LF and EF. Evidence has shown that PA also exists in its cleaved, active form in the serum, in complex with LF or EF (Mock, M. et al. 2003; Brossier, F. et al. 2001; Ezzell, J. W. et al. 1992). These complexes localize in lipid rafts within the cell membrane, and are subsequently endocytosed into acidic compartments, where the endosomal change in pH causes the PA components of the toxin to undergo a conformational change, forming a pore and releasing bound LF or EF into the cytosol, where they exert their effects directly on target proteins (Abrami, L. et al. 2003).

Until recently, LF had only one known group of natural substrates: the mitogen activated protein kinase kinase (MAPKK), or MEK, family of protein kinases, a central component of the MAP Kinase cascade that is involved in transcription of genes controlling the cell cycle, cell growth, differentiation, development, and multiple arms of the inflammatory response. LF specifically cleaves the MEKs at a consensus sequence near their amino termini, at which they normally bind to their own downstream targets, the mitogen activated protein kinase (MAPK), or ERK, family of protein kinases. Disruption of the MAPK signaling pathway by LF results in cell death via apoptosis in some cases (Rosenberger, C. M. et al. 2003), and down regulation of inflammatory biomarkers, leading to a general suppression of the innate immune response.

Recently, a second target of LF from the family of pathogen-associated molecular pattern (PAMP) recognition proteins known as the NOD-like receptors (NLRs) was discovered in rat and mouse macrophages (Hellmich, K. A. et al. 2012; Levinsohn, J. L. et al. 2012). Activation of these NLRs, specifically Nlrp1 in rats (Levinsohn, J. L. et al. 2012) and Nlrp1b in mice ((Hellmich, K. A. et al. 2012), occurs when LF cleaves a sequence of residues near their amino termini, as with the MAPKKs. Cleavage of Nlrp1/Nlrp1b causes it to oligomerize with other activated Nlrp1/Nlrp1b proteins, and to subsequently aid in the recruitment of caspase-1 to a multi-protein complex known as the inflammasome (Levinsohn, J. L. et al. 2012). Activation of caspase-1 by the inflammasome causes rapid programmed cell death, or pyroptosis, along with the production of inflammatory cytokines IL-1β and IL-1β (Hellmich, K. A. et al. 2012; Levinsohn, J. L. et al. 2012). Though this signaling pathway has not been characterized as extensively as the MAPK pathway, and the relationship between LF and Nlrp1 has thus far only been studied in rats and mice, humans express an ortholog of Nlrp1, and this role for LF may contribute to the rapid, systemic toxicity of anthrax infection that could not previously be explained by the cleavage of the MAPKKs alone.

The LF protein itself has four domains, which appear to have arisen through a series of duplications, mutations, and fusions over time (Pannifer, A. D. et al. 2001). Domain I is the protein's protective antigen-binding domain, which allows LF to complex with the carrier protein. Domains II, III, and IV together form a groove in which substrate binding and catalysis occur. Domain II resembles the ADP-ribosylating toxin of the closely related bacterium Bacillus cereus. Domain III folds into Domain II, and may be a product of a duplication of regions of Domain II. Domain IV is the catalytic domain of LF, a metalloproteinase domain classified as a member of the zincin family of zinc metalloproteinases, in the subfamily known as gluzincins (Spyroulias, G. A. et al. 2004). This domain also bears structural similarities to Domain I.

It is important to point out that the results of intoxication with LF have been shown to differ considerably from the systemic effects of clinical anthrax infection. Work done by Popov et al. (Popov et al. 2005) has shown that B. anthracis secretes a number of metalloproteinases in addition to LeTx, and these, as well as cell wall components released by dying bacteria, may play a role in the systemic effects of anthrax not seen when experimental animals are challenged with LeTx alone (Popov, S. G. et al. 2005; Supuran, C. T. et al. 2002). Nevertheless, LF may be responsible for the initial, primary physiological insult, incapacitating macrophages and dendritic cells within the lymph nodes, and allowing the bacteria to reach a threshold where they may then spread and cause these systemic effects, such as diffuse vasculitis, hemorrhagic lesions, and necrosis in multiple tissues (Brittingham, K. C. et al. 2005; Popov, S. G. et al. 2005). By limiting the initial insult through inhibition of LF, it may be possible to overcome the acute danger of anthrax, make treatment more effective, and decrease fatalities from infection.

Curcumin is a naturally occurring compound of the curcuminoid family, isolated originally from the plant Curcuma longa. The rhizome of this plant, specifically, is used to create the spice known as turmeric, and is a major component of the daily diet in many Asian countries. Even before the modern characterization of curcumin's molecular structure and functionality, it has long been used in traditional eastern medicines.

With its natural medicinal history in mind, curcumin has been studied extensively over the past few decades in a wide variety of systems, and has been found to exhibit significant pleiotropic effects. These effects may be attributed to the chemistry of curcumin, consisting of two polyphenolic rings joined by a conjugated, flexible linker region with a β-diketone moiety at its center (FIG. 1). The β-diketone moiety is capable of undergoing keto-enol tautomerization, though the enol form is more stable in both the solid phase and in solution (Gupta, S. C. et al. 2011) and is the dominant species at physiological pH (Gupta, S. C. et al. 2011; Zhang, Y. et al. 2012). The biological activities of curcumin are wide ranging: beyond having intrinsic antioxidant properties, it has been found to bind a wide spectrum of cellular constituents in vitro and in vivo, including inflammatory molecules, protein kinases, carrier proteins, cell survival proteins, structural proteins, the prion protein, antioxidant response elements, metal ions, and more (Gupta, S. C. et al. 2011). In addition, curcumin shows virtually no toxicity in humans (Gupta, S. C. et al. 2011; Ammon, H. P. T. et al. 1991).

### SUMMARY OF THE INVENTION

The present invention provides a method of inhibiting the binding of anthrax lethal factor with protective antigen comprising contacting the anthrax lethal factor with a compound having the structure:

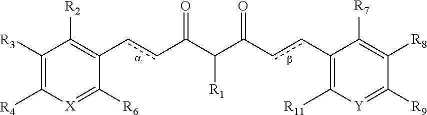

wherein

bond α and β are each, independently, present or absent;

X is CR₅ or N; Y is CR₁₀ or N;

R₁ is H, CF₃, halogen, —NO₂, —OCF₃, —OR₁₂, —NHCOR₁₂, —CONR₁₂R₁₃, —CSNR₁₂R₁₃, —C(═NH)NR₁₂R₁₃—SR₁₂, —SO₂R₁₃, —COR₁₄, —CSR₁₄, —C(═NR₁₂)R₁₄, —C(═NR₁₂)NR₁₃R₁₄, —SOR₁₂, —SONR₁₂R₁₃, —SO₂NR₁₂R₁₃, —P(O)R₁₂, —PH(═O)OR₁₂—P(═O)(OR₁₂)(OR₁₃), or —P(OR₁₂)(OR₁₃),
wherein R₁₂ and R₁₃ are each, independently, H, C₁₋₁₀ alkyl, C₂₋₁₀ alkenyl, C₂₋₁₀ alkynyl, aryl, heteroaryl, or heterocyclyl;
R₁₄ is C₂₋₁₀ alkyl, C₂₋₁₀ alkenyl, C₂₋₁₀ alkynyl, heteroaryl, heterocyclyl, methoxy, —OR₁₅, —NR₁₆R₁₇, or

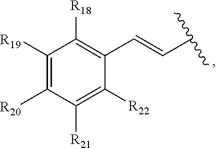

wherein R₁₅ is H, C₃₋₁₀ alkyl, C₂₋₁₀ alkenyl, C₂₋₁₂ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
R₁₈, R₁₉, R₂₁, and R₂₂ are each independently H, halogen, —NO₂, —CN, —NR₂₃R₂₄, —SR₂₃, —SO₂R₂₃, —CO₂R₂₃, —OR₂₅, CF₃, —SOR₂₃, —POR₂₃, —C(═S)R₂₃, —C(═NH)R₂₃, —C(═N)R₂₃, —P(═O)(OR₂₃)(OR₂₄), —P(OR₂₃)(OR₂₄), —C(═S)R₂₃, C₁₋₁₀ alkyl, C₂₋₁₀ alkenyl, C₂₋₁₀ alkynyl, aryl, heteroaryl, or heterocyclyl;
   wherein R₂₃, R₂₄, and R₂₅ are each, independently, H, C₁₋₁₀ alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
   wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl;

R₂, R₃, R₄, R₅, R₆, R₇, R₈, R₉, R₁₀, and R₁₁ are each independently, H, halogen, —NO₂, —CN, —NR₂₈R₂₉, —NHR₂₈R₂₉⁺, —SR₂₈, —SO₂R₂₈, —OR₂₈, —CO₂R₂₈, CF₃, C₁₋₁₀ alkyl, C₂₋₂₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and

wherein when R₁ is H, then R₃, R₄, R₅, R₈, R₉, or R₁₀, is halogen, —NO₂, —CN, —NR₂₈R₂₉, —NHR₂₈R₂₉⁺, —SR₂₈, —SO₂R₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and

wherein each occurrence of alkyl, alkenyl, or alkynyl is branched or unbranched, unsubstituted or substituted;

or a pharmaceutically acceptable salt thereof.

The present invention provides a method of treating a subject infected with Bacillus anthracis comprising administering to the subject a compound having the structure:

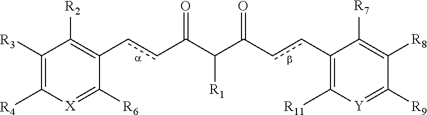

wherein

bond α and β are each, independently, present or absent;

X is CR₅ or N; Y is CR₁₀ or N;

R₁ is H, CF₃, halogen, —NO₂, —OCF₃, —OR₁₂, —NHCOR₁₂, —CONR₁₂R₁₃, —CSNR₁₂R₁₃, —C(═NH)NR₁₂R₁₃—SR₁₂, —SO₂R₁₃, —COR₁₄, —CSR₁₄, —C(═NR₁₂)R₁₄, —C(═NR₁₂)NR₁₃R₁₄, —SOR₁₂, —SONR₁₂R₁₃, —SO₂NR₁₂R₁₃, —P(O)R₁₂, —PH(═O)OR₁₂—P(═O)(OR₁₂)(OR₁₃), or —P(OR₁₂)(OR₁₃),
wherein R₁₂ and R₁₃ are each, independently, H, C₁₋₁₀ alkyl, C₂₋₁₀ alkenyl, C₂₋₁₀ alkynyl, aryl, heteroaryl, or heterocyclyl;
R₁₄ is C₂₋₁₀ alkyl, C₂₋₁₀ alkenyl, C₂₋₁₀ alkynyl, heteroaryl, heterocyclyl, methoxy, —OR₁₅, —NR₁₆R₁₇, or

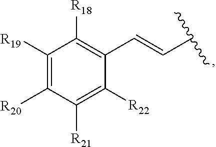

wherein R₁₅ is H, C₃₋₁₀ alkyl, C₂₋₁₀ alkenyl, C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
R₁₈, R₁₉, R₂₁, and R₂₂ are each independently H, halogen, —NO₂, —CN, —NR₂₃R₂₄, —SR₂₃, —SO₂R₂₃, —CO₂R₂₃, —OR₂₅, CF₃, —SOR₂₃, —POR₂₃, —C(═S)R₂₃, —C(═NH)R₂₃, —C(═N)R₂₃, —P(═O)(OR₂₃)(OR₂₄), —P(OR₂₃)(OR₂₄), —C(═S)R₂₃, C₁₋₁₀ alkyl, C₂₋₁₀ alkenyl, C₂₋₁₀ alkynyl, aryl, heteroaryl, or heterocyclyl;
   wherein R₂₃, R₂₄, and R₂₅ are each, independently, H, C₁₋₁₀ alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
   wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl;

R₂, R₃, R₄, R₅, R₆, R₇, R₈, R₉, R₁₀, and R₁₁ are each independently, H, halogen, —NO₂, —CN, —NR₂₈R₂₉, —NHR₂₈R₂₉⁺, —SR₂₈, —SO₂R₂₈, —OR₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and

wherein when R₁ is H, then R₃, R₄, R₅, R₈, R₉, or R₁₀, is halogen, —NO₂, —CN, —NR₂₈R₂₉, —NHR₂₈R₂₉⁺, —SR₂₈, —SO₂R₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and

wherein each occurrence of alkyl, alkenyl, or alkynyl is branched or unbranched, unsubstituted or substituted;

or a pharmaceutically acceptable salt thereof.

The present invention provides a pharmaceutical composition for use in inhibiting the binding of anthrax lethal factor with protective antigen, or for use in treating a subject infected with Bacillus anthracia, the pharmaceutical composition comprising the compound having the structure:

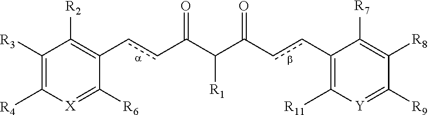

wherein

bond α and β are each, independently, present or absent;

X is CR₅ or N; Y is CR₁₀ or N;

R₁ is H, CF₃, halogen, —NO₂, —OCF₃, —OR₁₂, —NHCOR₁₂, —CONR₁₂R₁₃, —CSNR₁₂R₁₃, —C(═NH)NR₁₂R₁₃—SR₁₂, —SO₂R₁₃, —COR₁₄, —CSR₁₄, —C(═NR₁₂)R₁₄, —C(═NR₁₂)NR₁₃R₁₄, —SOR₁₂, —SONR₁₂R₁₃, —SO₂NR₁₂R₁₃, —P(O)R₁₂, —PH(═O)OR₁₂—P(═O)(OR₁₂)(OR₁₃), or —P(OR₁₂)(OR₁₃),
wherein R₁₂ and R₁₃ are each, independently, H, C₁₋₁₀ alkyl, C₂₋₁₀ alkenyl, C₂₋₁₀ alkynyl, aryl, heteroaryl, or heterocyclyl;
R₁₄ is C₂₋₁₀ alkyl, C₂₋₁₀ alkenyl, C₂₋₁₀ alkynyl, heteroaryl, heterocyclyl, methoxy, —OR₁₅, —NR₁₆R₁₇, or

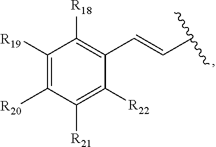

wherein R₁₅ is H, C₃₋₁₀ alkyl, C₂₋₁₀ alkenyl, C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
R₁₈, R₁₉, R₂₁, and R₂₂ are each independently H, halogen, —NO₂, —CN, —NR₂₃R₂₄, —SR₂₃, —SO₂R₂₃, —CO₂R₂₃, —OR₂₅, CF₃, —SOR₂₃, —POR₂₃, —C(═S)R₂₃, —C(═NH)R₂₃, —C(═N)R₂₃, —P(═O)(OR₂₃)(OR₂₄), —P(OR₂₃)(OR₂₄), —C(═S)R₂₃, C₁₋₁₀ alkyl, C₂₋₁₀ alkenyl, C₂₋₁₀ alkynyl, aryl, heteroaryl, or heterocyclyl;
   wherein R₂₃, R₂₄, and R₂₅ are each, independently, H, C₁₋₁₀ alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
   wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl;

R₂, R₃, R₄, R₅, R₆, R₇, R₈, R₉, R₁₀, and R₁₁ are each independently, H, halogen, —NO₂, —CN, —NR₂₈R₂₉, —NHR₂₈R₂₉⁺, —SR₂₈, —SO₂R₂₈, —OR₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and

wherein when R₁ is H, then R₃, R₄, R₅, R₈, R₉, or R₁₀, is halogen, —NO₂, —CN, —NR₂₈R₂₉, —NHR₂₈R₂₉⁺, —SR₂₈, —SO₂R₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and

wherein each occurrence of alkyl, alkenyl, or alkynyl is branched or unbranched, unsubstituted or substituted;

or a pharmaceutically acceptable salt thereof.

The present invention provides a compound for use in inhibiting the binding of anthrax lethal factor with protective antigen, or for use in treating a subject infected with Bacillus anthracis, the compound having the structure:

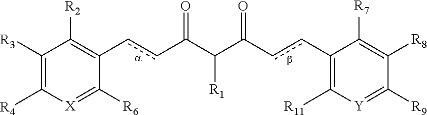

wherein

bond α and β are each, independently, present or absent;

X is CR₅ or N; Y is CR₁₀ or N;

R₁ is H, CF₃, halogen, —NO₂, —OCF₃, —OR₁₂, —NHCOR₁₂, —CONR₁₂R₁₃, —CSNR₁₂R₁₃, —C(═NH)NR₁₂R₁₃—SR₁₂, —SO₂R₁₃, —CSR₁₄, —C(═NR₁₂)R₁₄, —C(═NR₁₂)NR₁₃R₁₄, —SOR₁₂, —SONR₁₂R₁₃, —SO₂NR₁₂R₁₃, —P(O)R₁₂, —PH(═O)OR₁₂—P(═O)(OR₁₂)(OR₁₃), or —P(OR₁₂)(OR₁₃),
wherein R₁₂ and R₁₃ are each, independently, H, C₁₋₁₀ alkyl, C₂₋₁₀ alkenyl, C₂₋₁₀ alkynyl, aryl, heteroaryl, or heterocyclyl;
R₁₄ is C₂₋₁₀ alkyl, C₂₋₁₀ alkenyl, C₂₋₁₀ alkynyl, heteroaryl, heterocyclyl, methoxy, —OR₁₅, —NR₁₆R₁₇, or

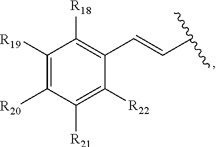

wherein R₁₅ is H, C₃₋₁₀ alkyl, C₂₋₁₀ alkenyl, C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
R₁₈, R₁₉, R₂₁, and R₂₂ are each independently H, halogen, —NO₂, —CN, —NR₂₃R₂₄, —SR₂₃, —SO₂R₂₃, —CO₂R₂₃, —OR₂₅, CF₃, —SOR₂₃, —POR₂₃, —C(═S)R₂₃, —C(═NH)R₂₃, —C(═N)R₂₃, —P(═O)(OR₂₃)(OR₂₄), —P(OR₂₃)(OR₂₄), —C(═S)R₂₃, C₁₋₁₀ alkyl, C₂₋₁₀ alkenyl, C₂₋₁₀ alkynyl, aryl, heteroaryl, or heterocyclyl;
   wherein R₂₃, R₂₄, and R₂₅ are each, independently, H, C₁₋₁₀ alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
   wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl;

R₂, R₃, R₄, R₅, R₆, R₇, R₈, R₉, R₁₀, and R₁₁ are each independently, H, halogen, —NO₂, —CN, —NR₂₈R₂₉, —NHR₂₈R₂₉⁺, —SR₂₈, —SO₂R₂₈, —OR₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and

wherein when R₁ is H, then R₃, R₄, R₅, R₈, R₉, or R₁₀, is halogen, —NO₂, —CN, —NR₂₈R₂₉, —NHR₂₈R₂₉⁺, —SR₂₈, —SO₂R₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and

wherein each occurrence of alkyl, alkenyl, or alkynyl is branched or unbranched, unsubstituted or substituted;

or a pharmaceutically acceptable salt thereof.

### BRIEF DESCRIPTION OF THE FIGURES

FIG. 1: Molecular structures of curcumin, CMC2.2, CMC2.24, CMC2.4, and CMC2.5.

FIG. 2A: Lethal factor activity as measured by fluorescence generated over time. 100 nM lethal factor, 3 μM substrate, and 20 μM inhibitors were used uniformly. Assay buffer was used in place of inhibitor as a control.

FIG. 2B: Percent residual activity of 100 nM lethal factor and 3 μM substrate after incubation with 20 μM curcumin, CMC2.2, CMC2.24, CMC2.4, or CMC2.5. R² values for all data exceeded 0.99, as determined by Levenberg-Marquardt linear regression analysis, indicating a high level of goodness of fit.

FIG. 3A: Dose-dependent inhibition of 100 nM lethal factor by curcumin at four substrate concentrations.

FIG. 3B: Dose-dependent inhibition of 100 nM lethal factor by CMC2.24 at four substrate concentrations.

FIG. 3C: Percent residual activity of 100 nM lethal factor at four concentrations of curcumin.

FIG. 3D: Percent residual activity of 100 nM lethal factor at four concentrations of CMC2.24.

FIG. 4A: Lineweaver-Burk and Dixon plots of 100 nM lethal factor activity over four substrate concentrations and four inhibitor concentrations: Lineweaver-Burk plot for curcumin.

FIG. 4B: Lineweaver-Burk and Dixon plots of 100 nM lethal factor activity over four substrate concentrations and four inhibitor concentrations: Lineweaver-Burk plot for CMC2.24.

FIG. 4C: Lineweaver-Burk and Dixon plots of 100 nM lethal factor activity over four substrate concentrations and four inhibitor concentrations: Dixon plot for curcumin.

FIG. 4D: Lineweaver-Burk and Dixon plots of 100 nM lethal factor activity over four substrate concentrations and four inhibitor concentrations: Dixon plot for CMC2.24.

FIG. 5A: Non-linear analysis of 100 nM lethal factor activity over four substrate concentrations and four inhibitor concentrations when data is fit to purely uncompetitive or purely non-competitive mechanism of inhibition: Curcumin fit to uncompetitive inhibition.

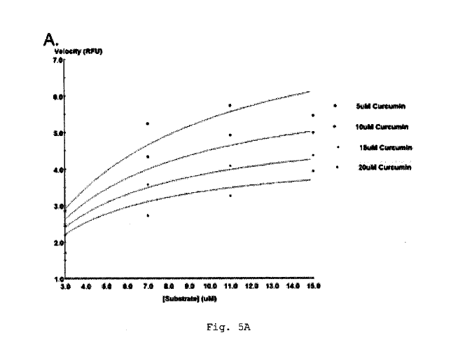

FIG. 5B: Non-linear analysis of 100 nM lethal factor activity over four substrate concentrations and four inhibitor concentrations when data is fit to purely uncompetitive or purely non-competitive mechanism of inhibition: CMC2.24 fit to uncompetitive inhibition.

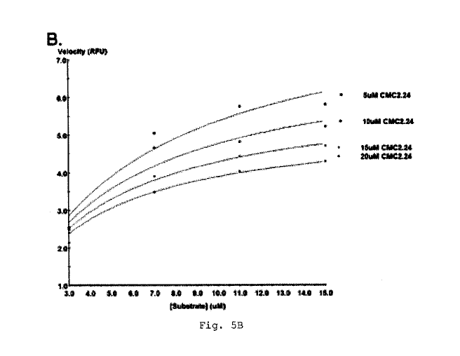

FIG. 5C: Non-linear analysis of 100 nM lethal factor activity over four substrate concentrations and four inhibitor concentrations when data is fit to purely uncompetitive or purely non-competitive mechanism of inhibition: Curcumin fit to non-competitive inhibition.

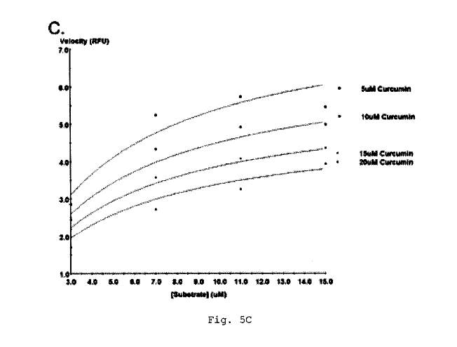

FIG. 5D: Non-linear analysis of 100 nM lethal factor activity over four substrate concentrations and four inhibitor concentrations when data is fit to purely uncompetitive or purely non-competitive mechanism of inhibition: CMC2.24 fit to non-competitive inhibition.

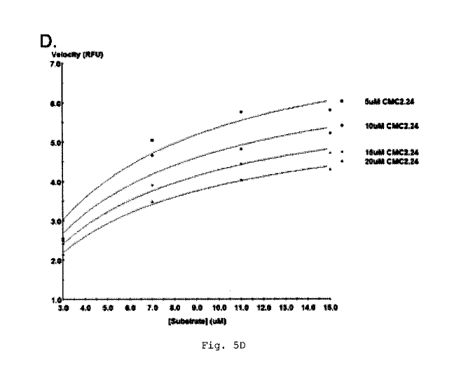

### DETAILED DESCRIPTION OF THE INVENTION

The present invention provides a method of inhibiting the binding of anthrax lethal factor with protective antigen comprising contacting the anthrax lethal factor with a compound having the structure:

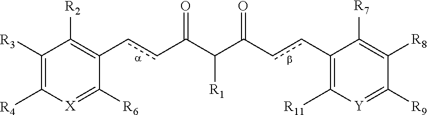

wherein

bond α and β are each, independently, present or absent;

X is CR₅ or N; Y is CR₁₀ or N;

R₁ is H, CF₃, halogen, —NO₂, —OCF₃, —OR₁₂, —NHCOR₁₂, —CONR₁₂R₁₃, —CSNR₁₂R₁₃, —C(═NH)NR₁₂R₁₃—SR₁₂, —SO₂R₁₃, —COR₁₄, —CSR₁₄, —C(═NR₁₂)R₁₄, —C(═NR₁₂)NR₁₃R₁₄, —SOR₁₂, —SONR₁₂R₁₃, —SO₂NR₁₂R₁₃, —P(O)R₁₂, —PH(═O)OR₁₂—P(═O)(OR₁₂)(OR₁₃), or —P(OR₁₂)(OR₁₃),
wherein R₁₂ and R₁₃ are each, independently, H, C₁₋₁₀ alkyl, C₂₋₁₀ alkenyl, C₂₋₁₀ alkynyl, aryl, heteroaryl, or heterocyclyl;
R₁₄ is C₂₋₁₀ alkyl, C₂₋₁₀ alkenyl, C₂₋₁₀ alkynyl, heteroaryl, heterocyclyl, methoxy, —OR₁₅, —NR₁₆R₁₇, or

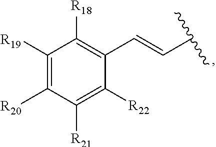

wherein R₁₅ is H, C₃₋₁₀ alkyl, C₂₋₁₀ alkenyl, C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
R₁₈, R₁₉, R₂₁, and R₂₂ are each independently H, halogen, —NO₂, —CN, —NR₂₃R₂₄, —SR₂₃, —SO₂R₂₃, —CO₂R₂₃, —OR₂₅, CF₃, —SOR₂₃, —POR₂₃, —C(═S)R₂₃, —C(═NH)R₂₃, —C(═N)R₂₃, —P(═O)(OR₂₃)(OR₂₄), —P(OR₂₃)(OR₂₄), —C(═S)R₂₃, C₁₋₁₀ alkyl, C₂₋₁₀ alkenyl, C₂₋₁₀ alkynyl, aryl, heteroaryl, or heterocyclyl;
   wherein R₂₃, R₂₄, and R₂₅ are each, independently, H, C₁₋₁₀ alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
   wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl;

R₂, R₃, R₄, R₅, R₆, R₇, R₈, R₉, R₁₀, and R₁₁ are each independently, H, halogen, —NO₂, —CN, —NR₂₈R₂₉, —NHR₂₈R₂₉⁺, —SR₂₈, —SO₂R₂₈, —OR₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and

wherein when R₁ is H, then R₃, R₄, R₅, R₈, R₉, or R₁₀, is halogen, —NO₂, —CN, —NR₂₈R₂₉, —NHR₂₈R₂₉⁺, —SR₂₈, —SO₂R₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and

wherein each occurrence of alkyl, alkenyl, or alkynyl is branched or unbranched, unsubstituted or substituted;

or a pharmaceutically acceptable salt thereof.

In some embodiments, the method wherein the compound inhibits anthrax lethal factor or anthrax lethal factor protease activity.

In some embodiments, the method wherein the compound is a non-competitive inhibitor of anthrax lethal factor.

In some embodiments, the method wherein the compound is an uncompetitive inhibitor of anthrax lethal factor.

The present invention provides a method of treating a subject infected with Bacillus anthracis comprising administering to the subject a compound having the structure:

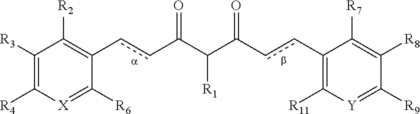

wherein

bond α and β are each, independently, present or absent;

X is CR₅ or N; Y is CR₁₀ or N;

R₁ is H, CF₃, halogen, —NO₂, —OCF₃, —OR₁₂, —NHCOR₁₂, —CONR₁₂R₁₃, —CSNR₁₂R₁₃, —C(═NH)NR₁₂R₁₃—SR₁₂, —SO₂R₁₃, —COR₁₄, —CSR₁₄, —C(═NR₁₂)R₁₄, —C(═NR₁₂)NR₁₃R₁₄, —SOR₁₂, —SONR₁₂R₁₃, —SO₂NR₁₂R₁₃, —P(O)R₁₂, —PH(═O)OR₁₂—P(═O)(OR₁₂)(OR₁₃), or —P(OR₁₂)(OR₁₃),
wherein R₁₂ and R₁₃ are each, independently, H, C₁₋₁₀ alkyl, C₂₋₁₀ alkenyl, C₂₋₁₀ alkynyl, aryl, heteroaryl, or heterocyclyl;
R₁₄ is C₂₋₁₀ alkyl, C₂₋₁₀ alkenyl, C₂₋₁₀ alkynyl, heteroaryl, heterocyclyl, methoxy, —OR₁₅, —NR₁₆R₁₇, or

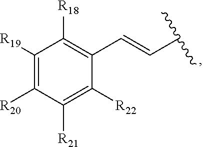

wherein R₁₅ is H, C₃₋₁₀ alkyl, C₂₋₁₀ alkenyl, C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
R₁₈, R₁₉, R₂₁, and R₂₂ are each independently H, halogen, —NO₂, —CN, —NR₂₃R₂₄, —SR₂₃, —SO₂R₂₃, —CO₂R₂₃, —OR₂₅, CF₃, —SOR₂₃, —POR₂₃, —C(═S)R₂₃, —C(═NH)R₂₃, —C(═N)R₂₃, —P(═O)(OR₂₃)(OR₂₄), —P(OR₂₃)(OR₂₄), —C(═S)R₂₃, C₁₋₁₀ alkyl, C₂₋₁₀ alkenyl, C₂₋₁₀ alkynyl, aryl, heteroaryl, or heterocyclyl;
   wherein R₂₃, R₂₄, and R₂₅ are each, independently, H, C₁₋₁₀ alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
   wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl;

R₂, R₃, R₄, R₅, R₆, R₇, R₈, R₉, R₁₀, and R₁₁ are each independently, H, halogen, —NO₂, —CN, —NR₂₈R₂₉, —NHR₂₈R₂₉⁺, —SR₂₈, —SO₂R₂₈, —OR₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and

wherein when R₁ is H, then R₃, R₄, R₅, R₈, R₉, or R₁₀, is halogen, —NO₂, —CN, —NR₂₈R₂₉, —NHR₂₈R₂₉⁺, —SR₂₈, —SO₂R₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and

wherein each occurrence of alkyl, alkenyl, or alkynyl is branched or unbranched, unsubstituted or substituted;

or a pharmaceutically acceptable salt thereof.

In some embodiments, the method wherein the compound inhibits anthrax lethal factor or anthrax lethal factor protease activity in the subject infected with Bacillus anthracis.

In some embodiments, the method wherein the compound inhibits the binding of anthrax lethal factor with protective antigen in the subject infected with Bacillus anthracis.

In some embodiments, the method wherein the compound inhibits the binding of anthrax edema factor with protective antigen in the subject infected with Bacillus anthracis.

In some embodiments, the method wherein the compound inhibits the formation of anthrax lethal toxin in the subject infected with Bacillus anthracis.

In some embodiments, the method wherein the compound inhibits the formation of anthrax edema toxin in the subject infected with Bacillus anthracis.

In some embodiments, the method further comprising administering an antibiotic to the subject infected with Bacillus anthracis.

In some embodiments, the method wherein the compound has the structure:

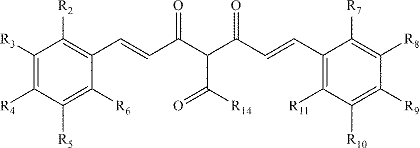

wherein R₁₄ is C₂₋₁₀ alkyl, C₂₋₁₀ alkenyl, C₂₋₁₀ alkynyl, heteroaryl, heterocyclyl, methoxy, —OR₁₅, —NR₁₆R₁₇, or

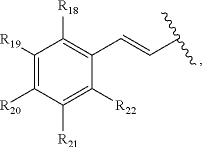

wherein R₁₅ is H, C₃₋₁₀ alkyl, C₂₋₁₀ alkenyl, C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
R₁₈, R₁₉, R₂₁, and R₂₂ are each independently H, halogen, —NO₂, —CN, —NR₂₃R₂₄, —SR₂₃, —SO₂R₂₃, —CO₂R₂₃, —OR₂₅, CF₃, C₁₋₁₀ alkyl, C₂₋₁₀ alkenyl, C₂₋₁₀ alkynyl, aryl, heteroaryl, or heterocyclyl;
   wherein R₂₃, R₂₄, and R₂₅ are each, independently, H, alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
   wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl;

R₂, R₃, R₄, R₅, R₆, R₇, R₈, R₉, R₁₀, and R₁₁ are each independently, H, halogen, —NO₂, —CN, —NR₂₈R₂₉, —SR₂₈, —SO₂R₂₈, —OR₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, or C₂₋₁₀ alkynyl; and

wherein each occurrence of alkyl, alkenyl, or alkynyl is branched or unbranched, unsubstituted or substituted; and

or a salt thereof.

In some embodiments, the method wherein at least one of R₂, R₃, R₄, R₅, and R₆ and at least one of R₇, R₈, R₉, R₁₀, and R₁₁, are each, independently, —OR₂₈.

In some embodiments, the method wherein
R₁₄ is methoxy, —OR₁₅ or —NR₁₆R₁₇;
R₁₅ is H, C₃₋₁₀ alkyl, C₂₋₁₀ alkenyl, or C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
or a salt thereof.

In some embodiments, the method wherein
R₁₄ is methoxy or —NR₁₆R₁₇;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
or a salt thereof.

In some embodiments, the method
wherein
R₁₄ is —OR₁₅,
R₁₅ is H, C₃₋₁₀ alkyl, C₂₋₁₀ alkenyl, or C₂₋₁₀ alkynyl;

or a salt thereof.

In some embodiments, the method
wherein
R₁₄ is —NR₁₆R₁₇,
   wherein R₁₆ and R₁₇ are each, independently, H or aryl;
R₂, R₃, R₄, R₅, R₆, R₇, R₈, R₉, R₁₀, and R₁₁ are each independently, H, —NR₂₈R₂₉, or —OR₂₈,
   wherein R₂₈ and R₂₉ are each, H or C₁₋₁₀ alkyl;

or a salt thereof.

In some embodiments, the method
wherein
R₁₄ is —NH-phenyl;
R₂, R₅, R₉, R₇, R₁₀, and R₁₁ are each H;
R₃, R₄, R₈, and R₉ are each, independently, H, —OH, or —OCH₃;

or a salt thereof.

In some embodiments, the method wherein the compound has the structure

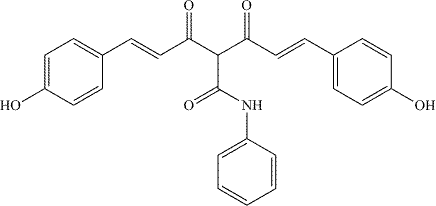 [SMILES: O=C(/C=C/c1ccc(O)cc1)C(C(=O)/C=C/c1ccc(O)cc1)C(=O)Nc1ccccc1]

or a pharmaceutically acceptable salt thereof.

In some embodiments, the method wherein the compound has the structure

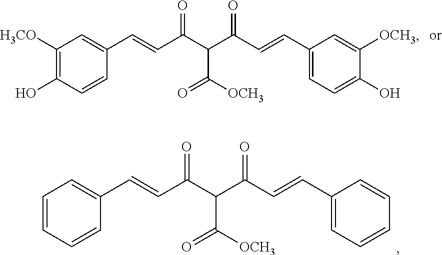 [SMILES: COC(=O)C(C(=O)/C=C/c1ccc(O)c(OC)c1)C(=O)/C=C/c1ccc(O)c(OC)c1 ; "or" ; COC(=O)C(C(=O)/C=C/c1ccccc1)C(=O)/C=C/c1ccccc1 ; ","]

or a pharmaceutically acceptable salt thereof.

The present invention provides a pharmaceutical composition for use in inhibiting the binding of anthrax lethal factor with protective antigen, or for use in treating a subject infected with Bacillus anthracis, the pharmaceutical composition comprising the compound having the structure:

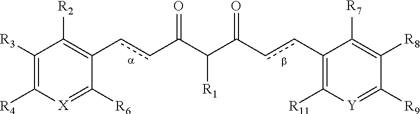

wherein

bond α and β are each, independently, present or absent;

X is CR₅ or N; Y is CR₁₀ or N;

R₁ is H, CF₃, halogen, —NO₂, —OCF₃, —OR₁₂, —NHCOR₁₂, —CONR₁₂R₁₃, —CSNR₁₂R₁₃, —C(═NH)NR₁₂R₁₃—SR₁₂, —SO₂R₁₃, —COR₁₄, —CSR₁₄, —C(═NR₁₂)R₁₄, —C(═NR₁₂)NR₁₃R₁₄, —SOR₁₂, —SONR₁₂R₁₃, —SO₂NR₁₂R₁₃, —P(O)R₁₂, —PH(═O)OR₁₂—P(═O)(OR₁₂)(OR₁₃), or —P(OR₁₂)(OR₁₃),
wherein R₁₂ and R₁₃ are each, independently, H, C₁₋₁₀ alkyl, C₂₋₁₀ alkenyl, C₂₋₁₀ alkynyl, aryl, heteroaryl, or heterocyclyl;
R₁₄ is C₂₋₁₀ alkyl, C₂₋₁₀ alkenyl, C₂₋₁₀ alkynyl, heteroaryl, heterocyclyl, methoxy, —OR₁₅, —NR₁₆R₁₇, or

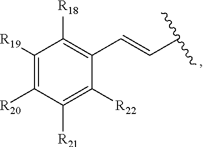

wherein R₁₅ is H, C₃₋₁₀ alkyl, C₂₋₁₀ alkenyl, C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
R₁₈, R₁₉, R₂₁, and R₂₂ are each independently H, halogen, —NO₂, —CN, —NR₂₃R₂₄, —SR₂₃, —SO₂R₂₃, —CO₂R₂₃, —OR₂₅, CF₃, —SOR₂₃, —POR₂₃, —C(═S)R₂₃, —C(═NH)R₂₃, —C(═N)R₂₃, —P(═O)(OR₂₃)(OR₂₄), —P(OR₂₃)(OR₂₄), —C(═S)R₂₃, C₁₋₁₀ alkyl, C₂₋₁₀ alkenyl, C₂₋₁₀ alkynyl, aryl, heteroaryl, or heterocyclyl;
   wherein R₂₃, R₂₄, and R₂₅ are each, independently, H, C₁₋₁₀ alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
   wherein R₂₆ and R₂₇ are each, independently, H, C₂₋₁₀ alkyl, C₂₋₁₀ alkenyl, C₂₋₁₀ alkynyl, aryl, heteroaryl, or heterocyclyl;

R₂, R₃, R₄, R₅, R₆, R₇, R₈, R₉, R₁₀, and R₁₁ are each independently, H, halogen, —NO₂, —CN, —NR₂₈R₂₉, —NHR₂₈R₂₉⁺, —SR₂₈, —SO₂R₂₈, —OR₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and

wherein when R₁ is H, then R₃, R₄, R₅, R₆, R₉, or R₁₀, is halogen, —NO₂, —CN, —NR₂₈R₂₉, —NHR₂₈R₂₉⁺, —SR₂₈, —SO₂R₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and

wherein each occurrence of alkyl, alkenyl, or alkynyl is branched or unbranched, unsubstituted or substituted;

or a pharmaceutically acceptable salt thereof.

The present invention provides a compound for use in inhibiting the binding of anthrax lethal factor with protective antigen, or for use in treating a subject infected with Bacillus anthracis, the compound having the structure:

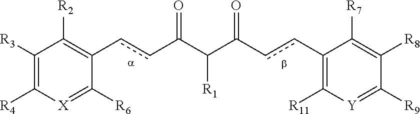

wherein

bond α and β are each, independently, present or absent;

X is CR₅ or N; Y is CR₁₀ or N;

R₁ is H, CF₃, halogen, —NO₂, —OCF₃, —OR₁₂, —NHCOR₁₂, —CONR₁₂R₁₃, —CSNR₁₂R₁₃, —C(═NH)NR₁₂R₁₃—SR₁₂, —SO₂R₁₃, —COR₁₄, —CSR₁₄, —C(═NR₁₂)R₁₄, —C(═NR₁₂)NR₁₃R₁₄, —SOR₁₂, —SONR₁₂R₁₃, —SO₂NR₁₂R₁₃, —P(O)R₁₂, —PH(═O)OR₁₂—P(═O)(OR₁₂)(OR₁₃), or —P(OR₁₂)(OR₁₃),
wherein R₁₂ and R₁₃ are each, independently, H, C₁₋₁₀ alkyl, C₂₋₁₀ alkenyl, C₂₋₁₀ alkynyl, aryl, heteroaryl, or heterocyclyl;
R₁₄ is C₂₋₁₀ alkyl, C₂₋₁₀ alkenyl, C₂₋₁₀ alkynyl, heteroaryl, heterocyclyl, methoxy, —OR₁₅, —NR₁₆R₁₇, or

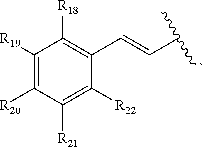

wherein R₁₅ is H, C₃₋₁₀ alkyl, C₂₋₁₀ alkenyl, C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
R₁₈, R₁₉, R₂₁, and R₂₂ are each independently H, halogen, —NO₂, —CN, —NR₂₃R₂₄, —SR₂₃, —SO₂R₂₃, —CO₂R₂₃, —OR₂₅, CF₃, —SOR₂₃, —POR₂₃, —C(═S)R₂₃, —C(═NH)R₂₃, —C(═N)R₂₃, —P(═O)(OR₂₃)(OR₂₄), —P(OR₂₃)(OR₂₄), —C(═S)R₂₃, C₁₋₁₀ alkyl, C₂₋₁₀ alkenyl, C₂₋₁₀ alkynyl, aryl, heteroaryl, or heterocyclyl;
   wherein R₂₃, R₂₄, and R₂₅ are each, independently, H, C₁₋₁₀ alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
   wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl;

R₂, R₃, R₄, R₅, R₆, R₇, R₈, R₉, R₁₀, and R₁₁ are each independently, H, halogen, —NO₂, —CN, —NR₂₈R₂₉, —NHR₂₈R₂₉⁺, —SR₂₈, —SO₂R₂₈, —OR₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and

wherein when R₁ is H, then R₃, R₄, R₅, R₈, R₉, or R₁₀, is halogen, —NO₂, —CN, —NR₂₈R₂₉, —NHR₂₈R₂₉⁺, —SR₂₈, —SO₂R₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and

wherein each occurrence of alkyl, alkenyl, or alkynyl is branched or unbranched, unsubstituted or substituted;

or a pharmaceutically acceptable salt thereof.

In an embodiment of the method, the compound wherein when R₁ is H, then R₃, R₄, R₅, R₈, R₉, or R₁₀, is —NO₂, —NR₂₈R₂₉, —NHR₂₈R₂₉⁺, —SR₂₈, —SO₂R₂₈, —CO₂R₂₈, CF₃, C₁₋₁₀ alkyl, C₂₋₁₀ alkenyl, C₂₋₁₀ alkynyl; or a salt thereof.

In an embodiment of the method, the compound wherein when R₁ is H, then R₄ or R₉ is —NO₂, —NR₂₈R₂₉, —NHR₂₈R₂₉⁺; or a salt thereof.

In an embodiment of the method, the compound wherein when R₁ is H, then R₄ or R₉ is —NR₂₈R₂₉ or —NHR₂₈R₂₉⁺; or a salt thereof.

In an embodiment of the method, the compound wherein

R₁ is H or —COR₁₄,
wherein R₁₄ is methoxy or —NH-phenyl;

R₂, R₅, R₆, R₇, R₁₀ and R₁₁ are each H;

R₃, R₄, R₈, and R₉ are each, independently H, —OH, —OCH₃, —N(CH₃)₂ or —NH(CH₃)₂⁺;

or a salt thereof.

In an embodiment of the method, the compound has the structure

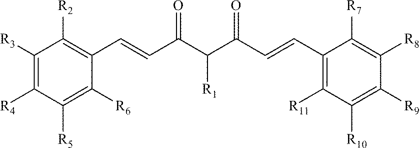

wherein R₁ is CF₃, halogen, —NO₂, —OCF₃, —OR₁₂, —NHCOR₁₂, —SR₁₂, —SO₂R₁₃, —COR₁₄, —CSR₁₄, or —CNR₁₄,
wherein R₁₂ and R₁₃ are each, independently, H, C₁₋₁₀ alkyl, C₂₋₁₀ alkenyl, C₂₋₁₀ alkynyl, aryl, heteroaryl, or heterocyclyl;
R₁₄ is C₂₋₁₀ alkyl, C₂₋₁₀ alkenyl, C₂₋₁₀ alkynyl, heteroaryl, heterocyclyl, methoxy, —OR₁₅, —NR₁₆R₁₇, or

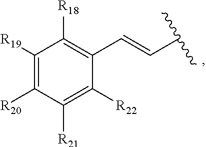

wherein R₁₅ is H, C₃₋₁₀ alkyl, C₂₋₁₀ alkenyl, C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
R₁₈, R₁₉, R₂₁, and R₂₂ are each independently H, halogen, —NO₂, —CN, —NR₂₃R₂₄, —SR₂₃, —SO₂R₂₃, —CO₂R₂₃, —OR₂₅, CF₃, C₁₋₁₀ alkyl, C₂₋₁₀ alkenyl, C₂₋₁₀ alkynyl, aryl, heteroaryl, or heterocyclyl;
   wherein R₂₃, R₂₄, and R₂₅ are each, independently, H, C₁₋₁₀ alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
   wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl;

R₂, R₃, R₄, R₅, R₆, R₇, R₈, R₉, R₁₀, and R₁₁ are each independently, H, halogen, —NO₂, —CN, —NR₂₈R₂₉, —SR₂₈, —SO₂R₂₈, —OR₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, or C₂₋₁₀ alkynyl; and

wherein each occurrence of alkyl, alkenyl, or alkynyl is branched or unbranched, unsubstituted or substituted; and

wherein at least one of R₂, R₃, R₄, R₅, and R₆ and at least one of R₇, R₈, R₉, R₁₀, and R₁₁, are each, independently, —OR₂₈;

or a salt thereof.

In yet another embodiment of the method, the compound has the structure

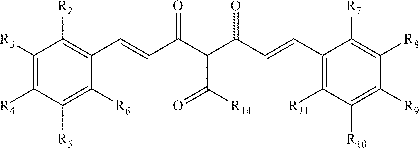

wherein R₁₄ is C₂₋₁₀ alkyl, C₂₋₁₀ alkenyl, C₂₋₁₀ alkynyl, heteroaryl, heterocyclyl, methoxy, —OR₁₅, —NR₁₆R₁₇, or

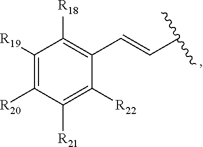

wherein R₁₅ is H, C₃₋₁₀ alkyl, C₂₋₁₀ alkenyl, C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
R₁₈, R₁₉, R₂₁, and R₂₂ are each independently H, halogen, —NO₂, —CN, —NR₂₃R₂₄, —SR₂₃, —SO₂R₂₃, —CO₂R₂₃, —OR₂₅, CF₃, C₁₋₁₀ alkyl, C₂₋₁₀ alkenyl, C₂₋₁₀ alkynyl, aryl, heteroaryl, or heterocyclyl;
   wherein R₂₃, R₂₄, and R₂₅ are each, independently, H, C₁₋₁₀ alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
   wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl;

R₂, R₃, R₄, R₅, R₆, R₇, R₈, R₉, R₁₀, and R₁₁ are each independently, H, halogen, —NO₂, —CN, —NR₂₈R₂₉, —SR₂₈, —SO₂R₂₈, —OR₂₈, —CO₂R₂₈, CF₃;
wherein R₂₈ and R₂₉ are each, H, CF₃, C₁₋₅ alkyl, C₂₋₅ alkenyl, or C₂₋₅ alkynyl; and

wherein each occurrence of alkyl, alkenyl, or alkynyl is branched or unbranched, unsubstituted or substituted; and

wherein at least one of R₂, R₃, R₄, R₅, and R₆ and at least one of R₇, R₈, R₉, R₁₀, and R₁₁, are each, independently, —OR₂₈;

or a salt thereof.

In an embodiment of the method, the compound has the structure

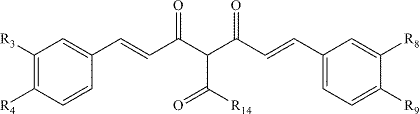

wherein R₃, R₄, R₈, and R₉ are H, —OCH₃, or —OH; R₁₄ is methoxy or —N(CH₃)₂; or a salt thereof.

In an embodiments of the method, the compound wherein R₁₄ is methoxy; R₂, R₃, R₄, R₅, R₆, R₇, R₈, R₉, R₁₀, and R₁₁ are each, independently, H, —OH, —OCH₃, —NO₂, or —CO₂CH₃; or a salt thereof.

In an embodiment of the method, the compound wherein X is N; or a salt thereof.

In an embodiment of the method, the compound wherein α and β are both present; or a salt thereof.

In some embodiments of the above methods, the compound has the structure

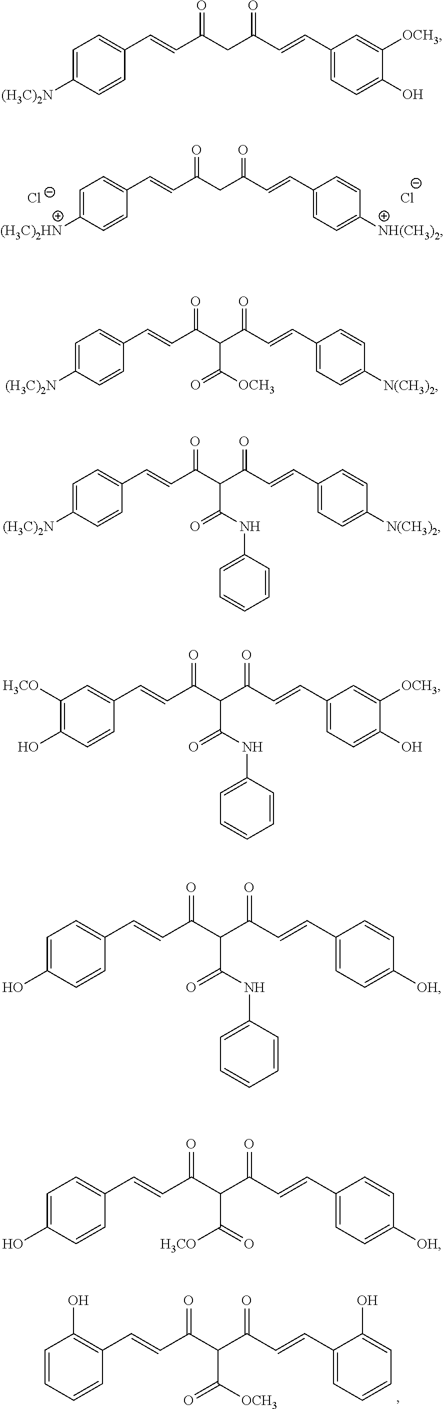

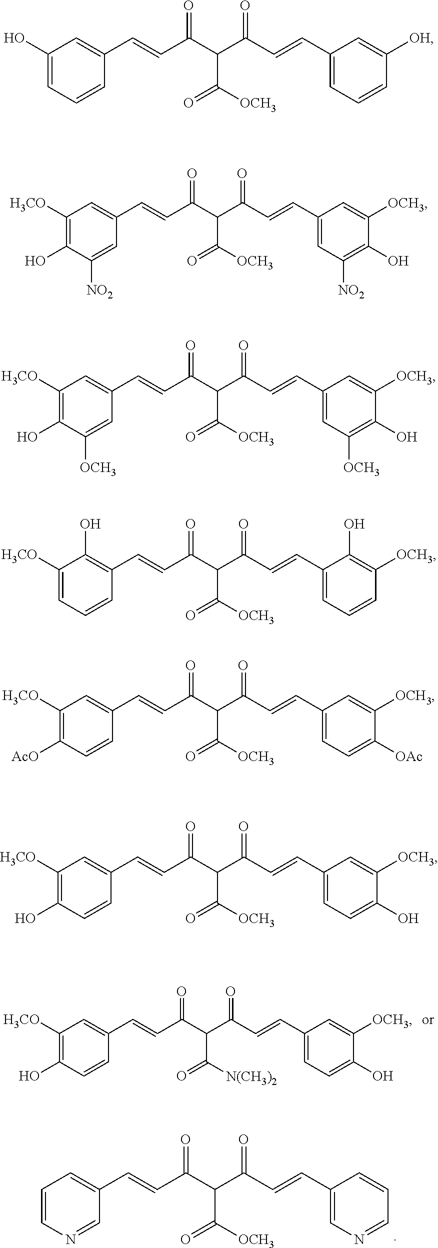

In some embodiments of the above methods, the compound is CMC2.24, which has the structure (ketonic form)

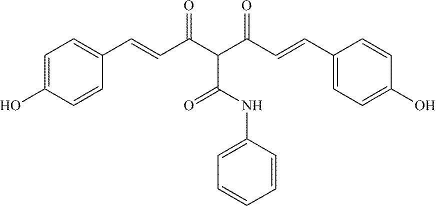 [SMILES: O=C(/C=C/c1ccc(O)cc1)C(C(=O)/C=C/c1ccc(O)cc1)C(=O)Nc1ccccc1]

or a pharmaceutically acceptable salt thereof.

In some embodiments, the method wherein compound has the structure

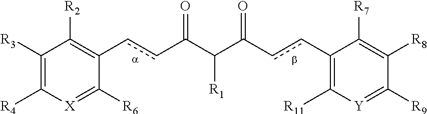

wherein

bond α and β are each, independently, present or absent;

X is CR₅ or N; Y is CR₁₀ or N;

R₁ is H, CF₃, halogen, —NO₂, —OCF₃, —OR₁₂, —NHCOR₁₂, —SR₁₂, —SO₂R₁₃, —COR₁₄, —CSR₁₄, —C(═NR₁₂)R₁₄, —C(═NH)R₁₄, —SOR₁₂, —POR₁₂, —P(═O)(OR₁₂)(OR₁₃), or —P(OR₁₂)(OR₁₃),
wherein R₁₂ is C₁₋₁₀ alkyl, C₂₋₁₀ alkenyl, C₂₋₁₀ alkynyl, aryl, heteroaryl, or heterocyclyl;
R₁₃ is H, C₁₋₁₀ alkyl, C₂₋₁₀ alkenyl, C₂₋₁₀ alkynyl, aryl, heteroaryl, or heterocyclyl;
R₁₄ is C₂₋₁₀ alkyl, C₂₋₁₀ alkenyl, C₂₋₁₀ alkynyl, heteroaryl, heterocyclyl, methoxy, —OR₁₅, —NR₁₆R₁₇, or

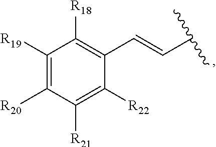

wherein R₁₅ is H, C₃₋₁₀ alkyl, C₂₋₁₀ alkenyl, C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
R₁₈, R₁₉, R₂₁, and R₂₂ are each independently H, halogen, —NO₂, —CN, —NR₂₃R₂₄, —SR₂₃, —SO₂R₂₃, —CO₂R₂₃, —OR₂₅, CF₃, —SOR₂₃, —POR₂₃, —C(═S)R₂₃, —C(═NH)R₂₃, —C(═NR₂₄)R₂₃, —C(═N)R₂₃, —P(═O)(OR₂₃)(OR₂₄), —P(OR₂₃)(OR₂₄), —C(═S)R₂₃, C₁₋₁₀ alkyl, C₂₋₁₀ alkenyl, C₂₋₁₀ alkynyl, aryl, heteroaryl, or heterocyclyl;
   wherein R₂₃, R₂₄, and R₂₅ are each, independently, H, C₁₋₁₀ alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
   wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl;

R₂, R₃, R₄, R₅, R₆, R₇, R₈, R₉, R₁₀, and R₁₁ are each independently, H, halogen, —NO₂, —CN, —NR₂₈R₂₉, —NHR₂₈R₂₉⁺, —SR₂₈, —SO₂R₂₈, —OR₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and

wherein when R₁ is H, then R₃, R₄, R₅, R₈, R₉, or R₁₀, is —CN, —SR₂₈, —SO₂R₂₈, —CO₂R₂₈, C₂₋₁₀ alkyl, C₂₋₁₀ alkenyl, C₂₋₁₀ alkynyl, aryl, heteroaryl, or heterocyclyl;

wherein R₂₈ and R₂₉ are each, H, CF₃, C₂₋₁₀ alkyl, C₂₋₁₀ alkenyl, C₂₋₁₀ alkynyl, or —C(═O)-heterocyclyl; or a salt thereof.

Chemically-modified curcumins possessing an electron-withdrawing group at the C-4 carbon and/or electron donating groups on the aryl rings are demonstrated to inhibit anthrax lethal factor and/or inhibit the binding of anthrax lethal factot with protective antigen.

In some embodiments, a method of treating a subject infected with Bacillus anthracis comprising administering to the subject the compound in an amount effective to treat the patient.

In some embodiments, a method of treating a subject infected with Bacillus anthracis comprising administering to the subject the compound in an amount effective to treat the patient in combination with an antibiotic.

Antibiotics which are detrimental to the bacterium Bacillus anthracis are within the scope of the method of this invention and include, but are not limited to, fluoroquinolines, tetracyclines, macrolides, glycopeptides or penicillins. In some embodiments, the antibiotic is ciprofloxacin, levofloxacin, erythromycin, vancomycin, doxycycline, or penicillin G. Two or more combined antibiotics may be used together or sequentially.

Variations on the following general synthetic methods (Pabon, H. 1964) will be readily apparent to those skilled in the art and are used to prepare the compounds of the method of the present invention.

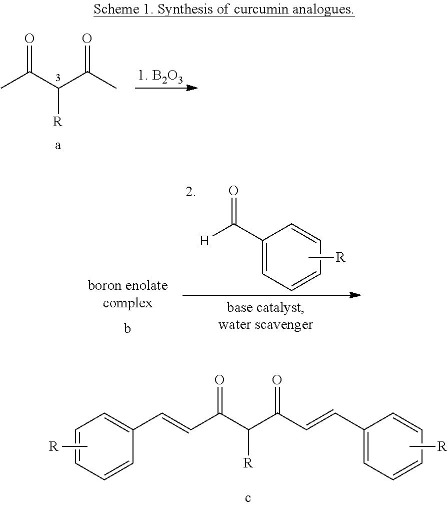

The synthesis of the curcumin analogues of the present invention can be carried out according to general Scheme 1. The R groups designate any number of generic substituents.

The starting material is provided by 2,4-pentanedione, which is substituted at the 3-carbon (see compound a). The desired substituted 2,4-pentanedione may be purchased from commercial sources or it may be synthesized using conventional functional group transformations well-known in the chemical arts, for example, those set forth in Organic Synthesis, Michael B. Smith, (McGraw-Hill) Second ed. (2001) and March's Advanced Organic Chemistry: Reactions, Mechanisms, and Structure, Michael B. Smith and Jerry March, (Wiley) Sixth ed. (2007), and specifically by Bingham and Tyman (45) and in the case of 3-aryl-aminocarbonyl compounds by Dieckman, Hoppe and Stein (46), the contents of which are hereby incorporated by reference. 2,4-pentanedione a is reacted with boron trioxide to form boron enolate complex b.

Boron enolate complex b is a complex formed by coordination of the enolate of compound a with boron. It is understood by those having ordinary skill in the art that the number of compound a enolates that may coordinate to boron as well as the coordination mode, i.e. monodentate versus bidentate, are variable so long as reaction, such as Knoevenagel condensation, at the C-3 carbon of the 2,4-pentanedione is suppressed.

Boron enolate complex b is then exposed to a benzaldehyde compound in the presence of a base catalyst and a water scavenger to form curcumin analogue c via aldol condensation. The ordinarily skilled artisan will appreciate that the benzaldehyde may possess various substituents on the phenyl ring so long as reactivity at the aldehyde position is not hindered. Substituted benzaldehyde compounds may be purchased from commercial sources or readily synthesized using aryl substitution chemistry that is well-known in the art. Suitable base catalysts for the aldol step include, but are not limited to, secondary amines, such as n-butylamine and n-butylamine acetate, and tertiary amines. Suitable water scavengers include, but are not limited to, alkyl borates, such as trimethyl borate, alkyl phosphates, and mixtures thereof. Other suitable reaction parameters have also been described by Krackov and Bellis in U.S. Pat. No. 5,679,864, the content of which is hereby incorporated by reference.

As used herein, the term “activity” refers to the activation, production, expression, synthesis, intercellular effect, and/or pathological or aberrant effect of the referenced molecule, either inside and/or outside of a cell. Such molecules include, but are not limited to, cytokines, enzymes, growth factors, pro-growth factors, active growth factors, and pro-enzymes. Molecules such as cytokines, enzymes, growth factors, pro-growth factors, active growth factors, and pro-enzymes may be produced, expressed, or synthesized within a cell where they may exert an effect. Such molecules may also be transported outside of the cell to the extracellular matrix where they may induce an effect on the extracellular matrix or on a neighboring cell. It is understood that activation of inactive cytokines, enzymes and pro-enzymes may occur inside and/or outside of a cell and that both inactive and active forms may be present at any point inside and/or outside of a cell. It is also understood that cells may possess basal levels of such molecules for normal function and that abnormally high or low levels of such active molecules may lead to pathological or aberrant effects that may be corrected by pharmacological intervention.

The compounds of the present invention include all hydrates, solvates, and complexes of the compounds used by this invention. If a chiral center or another form of an isomeric center is present in a compound of the present invention, all forms of such isomer or isomers, including enantiomers and diastereomers, are intended to be covered herein. Compounds containing a chiral center may be used as a racemic mixture, an enantiomerically enriched mixture, or the racemic mixture may be separated using well-known techniques and an individual enantiomer may be used alone. The compounds described in the present invention are in racemic form or as individual enantiomers. The enantiomers can be separated using known techniques, such as those described in Pure and Applied Chemistry 69, 1469-1474, (1997) IUPAC. In cases in which compounds have unsaturated carbon-carbon double bonds, both the cis (Z) and trans (E) isomers are within the scope of this invention.

The compounds of the subject invention may have spontaneous tautomeric forms. In cases wherein compounds may exist in tautomeric forms, such as keto-enol tautomers, each tautomeric form is contemplated as being included within this invention whether existing in equilibrium or predominantly in one form.

In the compound structures depicted herein, hydrogen atoms are not shown for carbon atoms having less than four bonds to non-hydrogen atoms. However, it is understood that enough hydrogen atoms exist on said carbon atoms to satisfy the octet rule.

This invention also provides isotopic variants of the compounds disclosed herein, including wherein the isotopic atom is ²H and/or wherein the isotopic atom ¹³C. Accordingly, in the compounds provided herein hydrogen can be enriched in the deuterium isotope. It is to be understood that the invention encompasses all such isotopic forms.

It is understood that the structures described in the embodiments of the methods hereinabove can be the same as the structures of the compounds described hereinabove.

It is understood that where a numerical range is recited herein, the present invention contemplates each integer between, and including, the upper and lower limits, unless otherwise stated.

Except where otherwise specified, if the structure of a compound of this invention includes an asymmetric carbon atom, it is understood that the compound occurs as a racemate, racemic mixture, and isolated single enantiomer. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, NY, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

In the compounds used in the method of the present invention, the substituents may be substituted or unsubstituted, unless specifically defined otherwise.

In the compounds used in the method of the present invention, alkyl, heteroalkyl, monocycle, bicycle, aryl, heteroaryl and heterocycle groups can be further substituted by replacing one or more hydrogen atoms with alternative non-hydrogen groups. These include, but are not limited to, halo, hydroxy, mercapto, amino, carboxy, cyano, carbamoyl and aminocarbonyl and aminothiocarbonyl.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present invention, one of ordinary skill in the art will recognize that the various substituents, i.e. R₁, R₂, etc. are to be chosen in conformity with well-known principles of chemical structure connectivity.

As used herein, “alkyl” includes both branched and straight-chain saturated aliphatic hydrocarbon groups having the specified number of carbon atoms and may be unsubstituted or substituted. Thus, C₁-Cₙ as in “C₁-Cₙ alkyl” is defined to include groups having 1, 2, . . . , n−1 or n carbons in a linear or branched arrangement. For example, C₁-C₆, as in “C₁-C₆ alkyl” is defined to include groups having 1, 2, 3, 4, 5, or 6 carbons in a linear or branched arrangement, and specifically includes methyl, ethyl, n-propyl, isopropyl, n-butyl, t-butyl, pentyl, hexyl, and octyl.

As used herein, “alkenyl” refers to a non-aromatic hydrocarbon radical, straight or branched, containing at least 1 carbon to carbon double bond, and up to the maximum possible number of non-aromatic carbon-carbon double bonds may be present, and may be unsubstituted or substituted. For example, “C₂-C₆ alkenyl” means an alkenyl radical having 2, 3, 4, 5, or 6 carbon atoms, and up to 1, 2, 3, 4, or 5 carbon-carbon double bonds respectively. Alkenyl groups include ethenyl, propenyl, butenyl and cyclohexenyl.

The term “alkynyl” refers to a hydrocarbon radical straight or branched, containing at least 1 carbon to carbon triple bond, and up to the maximum possible number of non-aromatic carbon-carbon triple bonds may be present, and may be unsubstituted or substituted. Thus, “C₂-C₆ alkynyl” means an alkynyl radical having 2 or 3 carbon atoms and 1 carbon-carbon triple bond, or having 4 or 5 carbon atoms and up to 2 carbon-carbon triple bonds, or having 6 carbon atoms and up to 3 carbon-carbon triple bonds. Alkynyl groups include ethynyl, propynyl and butynyl.

“Alkylene”, “alkenylene” and “alkynylene” shall mean, respectively, a divalent alkane, alkene and alkyne radical, respectively. It is understood that an alkylene, alkenylene, and alkynylene may be straight or branched. An alkylene, alkenylene, and alkynylene may be unsubstituted or substituted.

As used herein, “heteroalkyl” includes both branched and straight-chain saturated aliphatic hydrocarbon groups having the specified number of carbon atoms and at least 1 heteroatom within the chain or branch.

As used herein, “heterocycle” or “heterocyclyl” as used herein is intended to mean a 5- to 10-membered nonaromatic ring containing from 1 to 4 heteroatoms selected from the group consisting of O, N and S, and includes bicyclic groups. “Heterocyclyl” therefore includes, but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also encompassed by this definition.

As herein, “cycloalkyl” shall mean cyclic rings of alkanes of three to eight total carbon atoms, or any number within this range (i.e., cyclopropyl, cyclobutyl, cyclopentyl, cyclohexyl, cycloheptyl or cyclooctyl).

As used herein, “monocycle” includes any stable polyatomic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl.

As used herein, “bicycle” includes any stable polyatomic carbon ring of up to 10 atoms that is fused to a polyatomic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include phenyl, p-toluenyl (4-methylphenyl), naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring.

As used herein, the term “polycyclic” refers to unsaturated or partially unsaturated multiple fused ring structures, which may be unsubstituted or substituted.

The term “arylalkyl” refers to alkyl groups as described above wherein one or more bonds to hydrogen contained therein are replaced by a bond to an aryl group as described above. It is understood that an “arylalkyl” group is connected to a core molecule through a bond from the alkyl group and that the aryl group acts as a substituent on the alkyl group. Examples of arylalkyl moieties include, but are not limited to, benzyl(phenylmethyl), p-trifluoromethylbenzyl(4-trifluoromethylphenylmethyl), 1-phenylethyl, 2-phenylethyl, 3-phenylpropyl, 2-phenylpropyl and the like.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridizine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “alkylheteroaryl” refers to alkyl groups as described above wherein one or more bonds to hydrogen contained therein are replaced by a bond to an heteroaryl group as described above. It is understood that an “alkylheteroaryl” group is connected to a core molecule through a bond from the alkyl group and that the heteroaryl group acts as a substituent on the alkyl group. Examples of alkylheteroaryl moieties include, but are not limited to, —CH₂—(C₅H₄N), —CH₂—CH₂—(C₅H₄N) and the like.

The term “heterocycle” or “heterocyclyl” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

The alkyl, alkenyl, alkynyl, aryl, heteroaryl and heterocyclyl substituents may be substituted or unsubstituted, unless specifically defined otherwise. In the compounds of the present invention, alkyl, alkenyl, alkynyl, aryl, heterocyclyl and heteroaryl groups can be further substituted by replacing one or more hydrogen atoms with alternative non-hydrogen groups. These include, but are not limited to, halo, hydroxy, mercapto, amino, carboxy, cyano and carbamoyl.

As used herein, the term “halogen” refers to F, Cl, Br, and I.

The terms “substitution”, “substituted” and “substituent” refer to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropryl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy(phenylmethoxy) and p-trifluoromethylbenzyloxy(4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

As used herein, the term “electron-withdrawing group” refers to a substituent or functional group that has the property of increasing electron density around itself relative to groups in its proximity. Electron withdrawing property is a combination of induction and resonance. Electron withdrawal by induction refers to electron cloud displacement towards the more electronegative of two atoms in a σ-bond. Therefore, the electron cloud between two atoms of differing electronegativity is not uniform and a permanent state of bond polarization occurs such that the more electronegative atom has a slight negative charge and the other atom has a slight positive charge. Electron withdrawal by resonance refers to the ability of substituents or functional groups to withdraw electron density on the basis of relevant resonance structures arising from p-orbital overlap. Suitable electron-withdrawing groups include, but are not limited to, —CN, —CF₃, halogen, —NO₂, —OCF₃, —OR₁₂, —NHCOR₁₂, —SR₁₂, —SO₂R₁₃, —COR₁₄, —CSR₁₄, —CNR₁₄, —C(═NR₁₂)R₁₄, —C(═NH)R₁₄, —SOR₁₂, —POR₁₂, —P(═O)(OR₁₂)(OR₁₃), or —P(OR₁₂)(OR₁₃),
wherein R₁₂ and R₁₃ are each, independently, H, C₁₋₁₀ alkyl, C₂₋₁₀ alkenyl, C₂₋₁₀ alkynyl, aryl, heteroaryl, or heterocyclyl;
R₁₄ is C₂₋₁₀ alkyl, C₂₋₁₀ alkenyl, C₂₋₁₀ alkynyl, heteroaryl, heterocyclyl, methoxy, —OR₁₅, —NR₁₆R₁₇, or

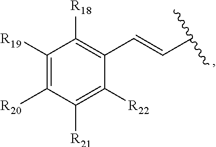

wherein R₁₅ is H, C₃₋₁₀ alkyl, C₂₋₁₀ alkenyl, C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
R₁₈, R₁₉, R₂₁, and R₂₂ are each independently H, halogen, —NO₂, —CN, —NR₂₃R₂₄, —SR₂₃, —SO₂R₂₃, —CO₂R₂₃, —OR₂₅, CF₃, —SOR₂₃, —POR₂₃, —C(═S)R₂₃, —C(═NH)R₂₃, C(═NR₂₄)R₂₃, —C(═N)R₂₃, —P(═O)(OR₂₃)(OR₂₄), —P(OR₂₃)(OR₂₄), —C(═S)R₂₃, C₁₋₁₀ alkyl, C₂₋₁₀ alkenyl, C₂₋₁₀ alkynyl, aryl, heteroaryl, or heterocyclyl;
   wherein R₂₃, R₂₄, and R₂₅ are each, independently, H, C₁₋₁₀ alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
   wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl.

While curcumin has been known to bind metal ions such as those of copper, iron, and zinc, affinity for zinc has been shown to be weak.

In the subject invention, the biological activity of curcumin analogues is attributed in part to their ability to access and bind zinc ions and an enhanced solubility. This invention describes that the enhancement of zinc binding affinity through the installation of electron-withdrawing and electron-donating groups at strategic locations, namely the C-4 carbon and the aryl rings, on the curcumin skeleton.

Without wishing to be bound by theory, it is believed that zinc binding affinity arises from increased stability of the curcumin enolate formed by removal of hydrogen from the C-4 carbon, which then proceeds to form a complex with zinc. The stability of a carbanion, including an enolate, is directly related to the acidity of the ionizable hydrogen, such as an enolic hydrogen. In general, the stability of an enolate increases with increasing acidity of the enolic hydrogen. Herein, the enolic hydrogen refers to the hydrogen atom connected to the C-4 carbon of the curcumin skeleton.

The acidity of the enolic hydrogen of curcumin and its analogues can be enhanced by incorporation of an electron-withdrawing group at the C-4 carbon. Substituents which delocalize negative charge will enhance acidity and stability of the resulting carbanion, such as an enolate. Again, without wishing to be bound by theory, it is believed that the electron-withdrawing group allows the negative charge of the enolate to be delocalized into the electron-withdrawing group, thereby stabilizing the enolate, enhancing its stability, and increasing its zinc binding affinity.

The electronic characteristics of the aryl rings of curcumin are also relevant for enhancing zinc binding affinity and biological activity. Electron-donating groups on the aryl portions of the curcumin skeleton improve its activity. The incorporation of such electron-donating groups on the aryl rings may affect one or more factors, including enhancement of water solubility and improvement of cation-pi interactions. Without wishing to be bound by theory, the installation of electron-donating groups on the aryl rings, in conjunction with the choice of C-4 electron-withdrawing group, is believed to increase electron polarization within the molecule such that intermolecular dipole-dipole forces with surrounding water molecules is enhanced, thereby increasing water solubility. Electron-donating groups may also increase water solubility by enhancing hydrogen-bonding interactions with surrounding water molecules. Furthermore, with respect to cation-pi interactions, it is believed that electron-donating groups increase electron density on the aryl rings, thereby enhancing the aryls' ability to recognize and/or bind to cations or cation-containing proteins.

The choice of electron-withdrawing groups on the C-4 carbon and the choice of electron-donating groups on the aryl rings may be chosen using techniques well known by the ordinarily skilled artisan. In general, the electron donating ability of common substituents suitable for use on the aryl rings can be estimated by their Hammett σ values. The Hammett σₚₐᵣₐ value is a relative measurement comparing the electronic influence of the substituent in the para position of a phenyl ring to the electronic influence of a hydrogen substituted at the para position. Typically for aromatic substituents in general, a negative Hammett σₚₐᵣₐ value is indicative of a group or substituent having an electron-donating influence on a pi electron system (i.e., an electron-donating group) and a positive Hammett σₚₐᵣₐ value is indicative of a group or substituent having an electron-withdrawing influence on a pi electron system (i.e., an electron-withdrawing group). Similarly, Hammett σₘₑₜₐ value is a relative measurement comparing the electronic influence of the substituent in the meta position of a phenyl ring to the electronic influence of a hydrogen substituted at the meta position. A list of Hammett σₚₐᵣₐ and σₘₑₜₐ values for common substituents can be found in Lowry and Richardson, “Mechanism and Theory in Organic Chemistry”, 3rd ed, p. 144. The effect of some substituents, including some electron-withdrawing groups, on C—H acidity can also be found on page 518 in Lowry and Richardson, “Mechanism and Theory in Organic Chemistry”, 3rd ed, the content of which is hereby incorporated by reference.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

In choosing the compounds of the present invention, one of ordinary skill in the art will recognize that the various substituents, i.e. R₁, R₂, etc. are to be chosen in conformity with well-known principles of chemical structure connectivity.

The various R groups attached to the aromatic rings of the compounds disclosed herein may be added to the rings by standard procedures, for example those set forth in Advanced Organic Chemistry: Part B: Reaction and Synthesis, Francis Carey and Richard Sundberg, (Springer) 5th ed. Edition. (2007), the content of which is hereby incorporated by reference.

The compounds used in the method of the present invention may be prepared by techniques well known in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

Another aspect of the invention comprises a compound used in the method of the present invention as a pharmaceutical composition.

In some embodiments, a pharmaceutical composition comprising the compound of the present invention and a pharmaceutically acceptable carrier.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department Of Health And Human Services, 30^{th} edition, 2010), which are hereby incorporated by reference. Pharmaceutically active agents which have pendant carboxylic acid groups may be modified in accordance with the present invention using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present invention may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The compounds of the present invention may also form salts with basic amino acids such a lysine, arginine, etc. and with basic sugars such as N-methylglucamine, 2-amino-2-deoxyglucose, etc. and any other physiologically non-toxic basic substance.

As used herein, “treating” means preventing, slowing, halting, or reversing the progression of a disease or infection. Treating may also mean improving one or more symptoms of a disease or infection.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier as are slow-release vehicles.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise a single compound or mixtures thereof with additional antitumor agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or topically onto a site of disease or lesion, all using dosage forms well known to those of ordinary skill in the pharmaceutical arts.

The compounds used in the method of the present invention can be administered in admixture with suitable pharmaceutical diluents, extenders, excipients, or in carriers such as the novel programmable sustained-release multi-compartmental nanospheres (collectively referred to herein as a pharmaceutically acceptable carrier) suitably selected with respect to the intended form of administration and as consistent with conventional pharmaceutical practices. The unit will be in a form suitable for oral, nasal, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as small unilamellar vesicles, large unilamellar vesicles, and multilamellar vesicles. Liposomes can be formed from a variety of phospholipids such as lecithin, sphingomyelin, proteolipids, protein-encapsulated vesicles or from cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylaspartamidephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar-coated or film-coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, asuitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials such as solutol and/or ethanol to make them compatible with the type of injection or delivery system chosen.

The compounds and compositions of the present invention can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by topical administration, injection or other methods, to the afflicted area, such as a wound, including ulcers of the skin, all using dosage forms well known to those of ordinary skill in the pharmaceutical arts.

Specific examples of pharmaceutically acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

The term “prodrug” as used herein refers to any compound that when administered to a biological system generates the compound of the invention, as a result of spontaneous chemical reaction(s), enzyme catalyzed chemical reaction(s), photolysis, and/or metabolic chemical reaction(s). A prodrug is thus a covalently modified analog or latent form of a compound of the invention.

The active ingredient can be administered orally in solid dosage forms, such as capsules, tablets, powders, and chewing gum; or in liquid dosage forms, such as elixirs, syrups, and suspensions, including, but not limited to, mouthwash and toothpaste. It can also be administered parentally, in sterile liquid dosage forms.

Solid dosage forms, such as capsules and tablets, may be enteric-coated to prevent release of the active ingredient compounds before they reach the small intestine. Materials that may be used as enteric coatings include, but are not limited to, sugars, fatty acids, proteinaceous substances such as gelatin, waxes, shellac, cellulose acetate phthalate (CAP), methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), and methyl methacrylate-methacrylic acid copolymers.

The compounds and compositions of the invention can be coated onto stents for temporary or permanent implantation into the cardiovascular system of a subject.

The compounds of the present invention can be synthesized according to methods described in PCT International Publication No. WO 2010/132815 A9. Variations on those general synthetic methods will be readily apparent to those of ordinary skill in the art and are deemed to be within the scope of the present invention.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

Experimental Details

Materials and Methods

Abbreviations

CMC, chemically modified curcumin; EF, edema factor; ETx, edema toxin; LeTx, lethal toxin; LF, lethal factor; PA, protective antigen; MAPK, mitogen activated protein kinase; MAPKK, mitogen activated protein kinase kinase.

Materials

Recombinant lethal factor (native sequence) was purchased from List Biological Laboratories, Inc. (Campbell, Calif.), and used without further purification. The lyophilized enzyme was solubilized to 11.1 μM in 5 mM HEPES, 50 mM NaCl, and 1 mg/mL bovine serum albumin, at pH 7.5, and stored at −80° C., as per the manufacturer's protocol. MAPKKide, a FRET-quenched synthetic peptide analogous to the MAPKK N-terminal domain was also purchased in lyophilized form from List Biological Laboratories, Inc., solubilized to 5 mM in ≧99.9% A.C.S. spectrophotometric grade DMSO from Sigma-Aldrich, and stored at −20° C. without further purification, as per the manufacturer's protocol. Curcumin (99% purity) was purchased from Selleck Chemicals (Houston, Tex.), diluted to 50 mM in ≧99.9% DMSO (Sigma-Aldrich), and stored at −20° C. All chemically modified curcumin derivatives were synthesized as reported and characterized in a previously published paper (Zhang Y. et al. 2012), solubilized to 50 mM in ≧99.9% DMSO (Sigma-Aldrich), and stored at −20° C. Purity of ≧99.5% was ensured for all synthesized compounds through ¹H NMR, mass spectrometry, TLC, and HPLC. The reaction buffer prepared for use in all assays was 20 mM HEPES with 0.125 mg/mL bovine serum albumin, adjusted to pH 7.4, and stored at 4° C.

Lethal Factor Peptidolytic Activity Assay

Cleavage of the synthetic MAPKK substrate by LF results in separation of the donor and acceptor moieties and restoration of fluorescence. Development of fluorescence as read by a SpectraMax M2 multiwell microplate fluorometer (Ex/Em=490 nm/523 nm) was used to measure the rate of LF-catalyzed substrate cleavage.¹⁴ All reagents were diluted as necessary for each assay just prior to use. Reactions were carried out in triplicate, in 96-well microplates, at 37° C., with a final volume of 150 μL in each well: 50 μL of lethal factor, 50 μL of substrate, and 50 μL of inhibitor (or reaction buffer for control wells). LF was diluted with reaction buffer to a 100 nM final concentration in the well under all reaction conditions, while inhibitor and substrate were diluted to various concentrations, in reaction buffer, as described in the results. All LF and inhibitor/control pairs were incubated at 37° C. in the reaction wells for 20 minutes prior to the addition of substrate, and read by the fluorometer immediately after the addition of substrate. IC₅₀ values for curcumin and CMC2.24 were determined empirically by plotting the percentage of inhibition versus inhibitor concentration. Additional graphical analysis was performed using nonlinear curve fitting software (EnzFitter, Elsevier-Biosoft).

### EXAMPLE 1

### Dose Dependent Inhibition of LF Peptidolytic Activity

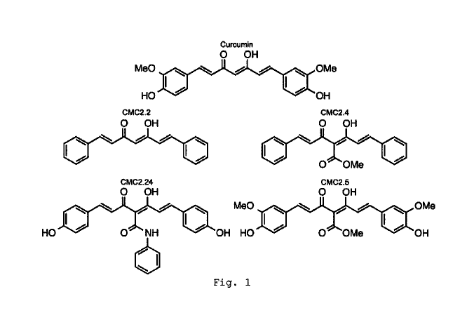

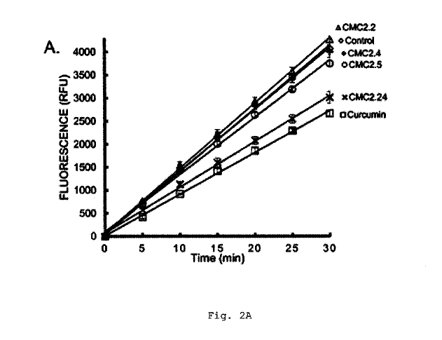

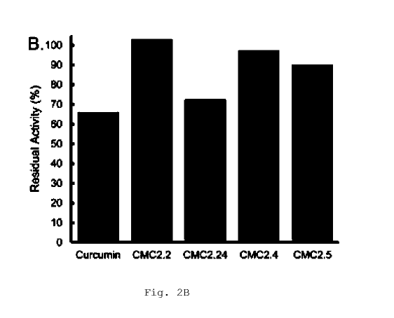

Curcumin and the CMCs (FIG. 1) were tested and found to inhibit lethal factor peptidolytic activity in a dose-dependent fashion with the exception of CMC2.2 (FIG. 2A-2B). The linear time course of the reactions indicates that neither the assay conditions nor the extended presence of the inhibitor had any denaturing effect on the enzyme, and thus that the data acquired does not reflect progressive irreversible inactivation.

### EXAMPLE 2

### Dose Dependant Inhibition of LF Peptidolytic Activity at Four Substrate Concentrations

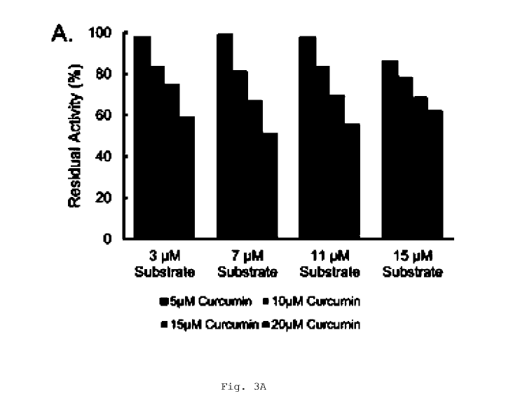

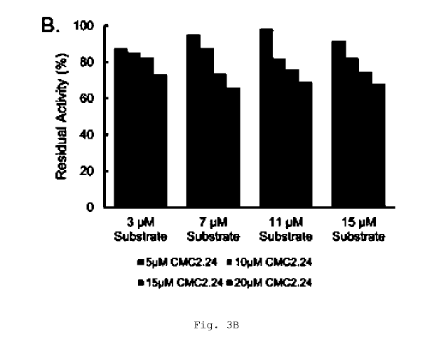

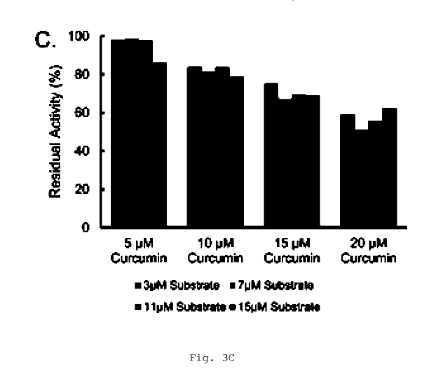

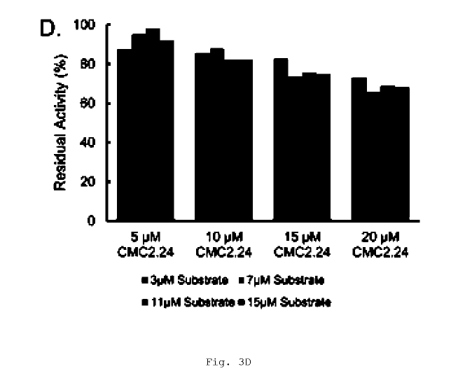

On average, in the presence of 20 μM curcumin, cleavage of 3 μM substrate with 100 nM lethal factor was inhibited by 33-41% (FIG. 3A-3D). Under the same conditions, CMC2.24 was capable of inhibiting LF to a comparable extent, resulting in 26-28% inhibition. The other CMCs exhibited somewhat weaker inhibitory potency, likely indicative of their specific chemical modifications (i.e. the removal of 3′ methoxy and 4′ hydroxyl groups for CMC2.2 and CMC2.4, believed to be important to binding). In the presence of CMC2.2, a derivative of curcumin stripped of nearly all functional groups, the level of LF activity was equal to or greater than that observed in the absence of any curcuminoids. IC₅₀ values for curcumin and CMC2.24 with 100 nM LF and 3 μM substrate were determined to be approximately 23.8 μM and 33.1 μM respectively, though it is worth noting that these values are significantly dependent on substrate concentration.

The placement of an electron-withdrawing group at the C-4 carbon of curcumin and curcumin analogues confers several advantages, such as improved water solubility, improved metal binding ability, and improved biological activity when compared to curcumin. Without wishing to be bound by theory, the presence of an electron-withdrawing group at the C-4 position of curcumin and curcumin analogues stabilizes the enol form of the compound as well as the enolate formed from deprotonation at the C-4 carbon, thereby facilitating water solubility and chelation of metal cations, such as Zn²⁺, by the resulting enolate. Accordingly, other curcumin analogues having electron-withdrawing groups at the C-4 carbon possess such properties and function in a similar manner to CMC2.24.

The installation of electron-withdrawing and/or electron-donating groups at strategic locations, namely the C-4 carbon and/or the aryl rings, on the curcumin skeleton results in improved properties. Curcumin analogs without the electron-withdrawing and/or electron-donating groups at strategic locations are not active or are significantly less active in inhibiting anthrax lethal factor and/or treating a subject infected with Bacillus anthracia.

### EXAMPLE 3

### Mechanistic Analysis of Inhibition

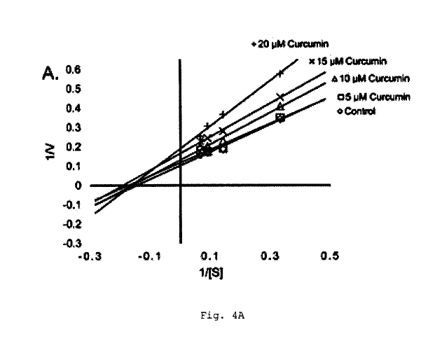

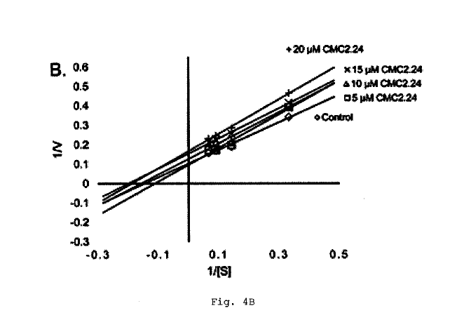

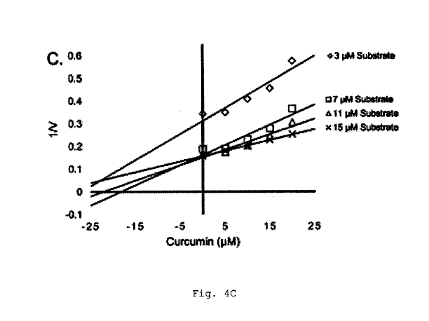

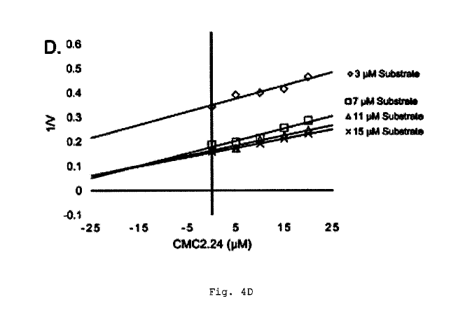

As the strongest inhibitor of lethal factor among the CMCs tested, CMC2.24 was assayed further, along with curcumin itself, in an attempt to begin to elucidate their mechanisms of action and potency. The same trend of dose-dependent inhibition was observed across four substrate concentrations tested for both curcumin and CMC2.24 (FIG. 4A-4D), and unexpectedly, the extent of inhibition of LF after incubation with either curcumin or CMC2.24 was unaffected or increased, with some variability across the spectrum of inhibitor concentrations, at increasing substrate concentrations (FIG. 4A-4D), suggesting that these compounds do not act through a simple competitive mechanism of inhibition, but rather that the curcuminoids act to inhibit LF through a mixture of inhibitory mechanisms. An increase in inhibition as a result of increasing substrate concentration, considered alone, would be evidence for uncompetitive inhibition; however the fact that this trend is not entirely consistent indicates that multiple modes of inhibition are likely to account for the reduced enzyme activity, rather than a single classical inhibitory mechanism. The linearity of the progress curves for extent of substrate cleavage over time (FIG. 2A-2B) suggests that the inhibitors are in rapidly reversible equilibrium with the enzyme.

Analysis of Lineweaver-Burk and Dixon plots (FIG. 4A-4D) also supported the hypothesis that these compounds do not inhibit lethal factor through a competitive mechanism, and as would be expected for a mixture of inhibitory mechanisms, the data fails to fit any one of the simple inhibitory patterns alone. Intersection of the lines in the second and third quadrant of a Lineweaver-Burk plot is an indication that curcumin and CMC2.24 may actually act to increase the affinity of LF for substrate while they decrease catalytic activity. This again is strong evidence against competitive inhibition, as it would be unlikely for a compound to increase an enzyme's affinity for substrate and to occupy its substrate binding site simultaneously without postulating multiple inhibitor binding sites on the enzyme. Instead, it appears that curcumin and CMC2.24 act through mechanisms of both uncompetitive and non-competitive inhibition, with the relative contributions of these two modes of inhibition being functions of both substrate and inhibitor concentrations.

If the inhibitory mechanisms of these compounds were that of pure non-competitive inhibition, a Lineweaver-Burk plot should produce lines that intersect on the X-axis, while pure uncompetitive inhibition should produce parallel lines. Neither of these patterns was observed exclusively for curcumin or CMC2.24, though it does appear that the lines are more parallel within the range of lower inhibitor concentrations for curcumin, and have a more parallel appearance overall for CMC2.24, indicative of a predominantly uncompetitive inhibitory mode for the latter. Families of Dixon Plots for both curcumin and CMC2.24 appeared as lines that intersect at a common point above the X-axis, as is seen for uncompetitive inhibition, with the exception of the lowest substrate concentration of both inhibitors assayed (3 μM), which deviates substantially from the trend. This provides additional support for the hypothesis that there are different inhibitory mechanisms at work over lower or higher relative substrate and inhibitor concentrations. Together, these patterns provide evidence that curcumin and CMC2.24 may have more than one mode of binding to lethal factor: one with a higher binding affinity, and a second with a lower binding affinity that does not play a substantial role until a threshold concentration of inhibitor is reached.

The hypothesis that curcumin and CMC2.24 act through a mixture of inhibitory mechanisms is not easily submitted to rigorous quantitation, though the preliminary data was analyzed according to models of pure non-competitive and pure uncompetitive inhibition, using nonlinear curve fitting algorithms. Treating curcumin as a purely uncompetitive inhibitor produced an apparent Kᵢ of 11.5 μM, while treating it as a purely non-competitive inhibitor produced an apparent Kᵢ of 20.3 μM-nearly double that of the uncompetitive fit. Treating CMC2.24 as a purely uncompetitive inhibitor produced an apparent Kᵢ of 19.7 μM, while treating it as a purely non-competitive inhibitor produced an apparent Kᵢ of 34.5 μM. These values suggest that aspects of both non-competitive and uncompetitive inhibition may play a part in the inhibition of lethal factor activity by curcumin and CMC2.24, and more specifically, that the compounds may be capable of binding both the enzyme-substrate complex (ES complex) and the free enzyme, but they appear to bind more tightly (nearly two-fold in the case of curcumin) to the ES complex, providing an explanation for the ratio of different inhibitory characteristics seen at varying substrate and inhibitor concentrations. Based on the preliminary data alone, however, it is still unclear whether these compounds actually bind to multiple sites on the enzyme, affecting substrate affinity and catalytic activity differentially at each of these putative sites.

### EXAMPLE 4

### Anthrax Lethal Toxin

An additional aspect of the invention provides the compound of Formula I that inhibits the binding of anthrax lethal factor with protective antigen, thereby preventing the formation of anthrax lethal toxin in a subject infected with Bacillus anthracis. An additional aspect of the invention provides the compound CMC2.24 that inhibits the binding of anthrax lethal factor with protective antigen, thereby preventing the formation of anthrax lethal toxin in a subject infected with Bacillus anthracis

### EXAMPLE 5

### CMC2.24 in Vivo Studies

An amount of a compound of Formula I is administered to a subject infected with Bacillus anthracis. The amount of the compound is effective to treat the subject. An amount of CMC2.24 is administered to a subject infected with Bacillus anthracis. The amount of CMC2.24 is effective to treat the subject.

### EXAMPLE 6

### Analogs of CMC2.24

An additional aspect of the invention provides analogs of the compound CMC2.24 that are active as inhibitors of lethal factor or protective antigen and/or prevent the formation of anthrax lethal toxin and/or edema toxin. Additional compounds (Scheme 2) have been manufactured as described in PCT International Application WO 2010/132815 A9, the contents of which are hereby incorporated by reference. The analogs of CMC2.24 shown below in Scheme 2 have analogous activity to CMC2.24.

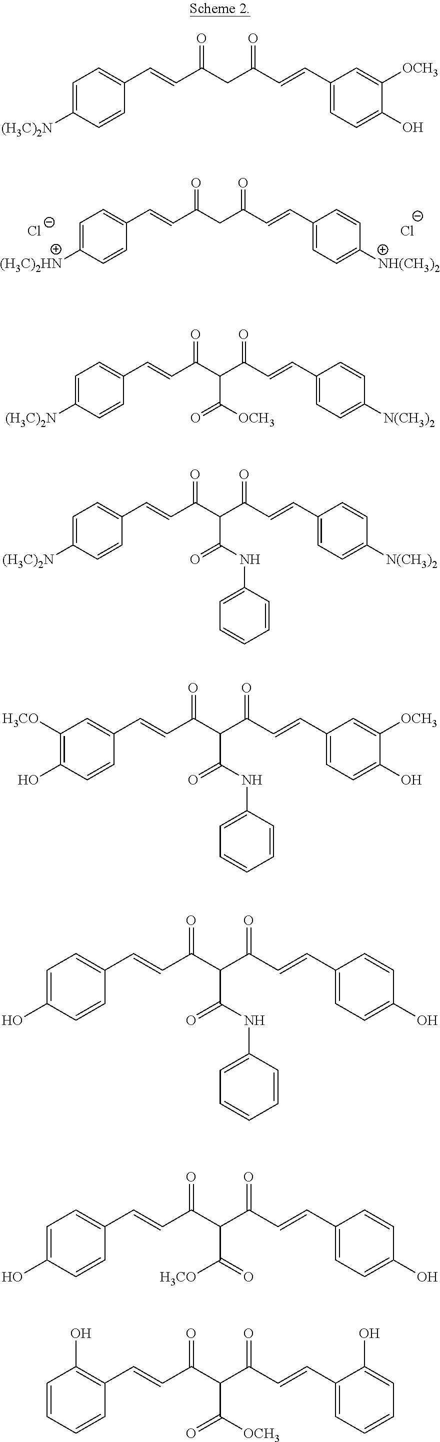

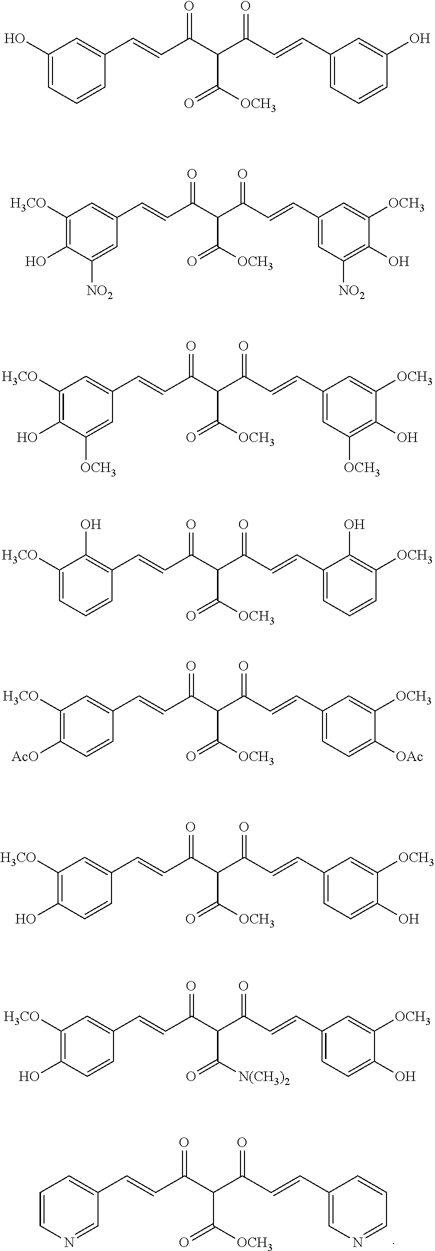

Discussion

The structural homology among the bacterial metalloproteinases, including LF, botulinum toxin, and thermolysin, as well as the eukaryotic matrix metalloproteinases, prompted consideration of whether inhibitors of the mammalian zinc-dependent proteolytic enzymes could also be used to inhibit the bacterial proteinases. Initial studies on compounds that inhibit both families of metalloproteinases were carried out by Kocer at al. using nonantimicrobial tetracyclines (Kocer, S. S. et al. 2005), and showed that these chemically modified tetracyclines were capable of inhibiting LF in human macrophages and monocytoid cell lines when administered before or even hours after the addition of the toxin to the cells. Given that the β-diketone moiety seems to be responsible for the tetracyclines' inhibition of other metalloproteinases via zinc binding (Zhang, Y. et al. 2012), the metal-chelating capacity of curcumin provided a basis for consideration that the compound could also inhibit these metalloenzymes by binding to the zinc atom in their active site through its own β-diketone moiety. In addition to the tetracyclines, other polyphenolic compounds, such as the catechin family of compounds, have also been shown to act as LF inhibitors (Dell'Acia, I. et al. 2004). The presence of polyphenolic groups in many of the known inhibitors of LF suggest that conjugated hydroxyl or oxo groups may play an important role in the binding of these compounds to the enzyme.

Chemically Modified Curcumins

Curcumin has shown promise as a platform for the development of drugs to target many diseases and syndromes, including cancer and inflammatory diseases, as well as anthrax; however, one of the major obstacles to overcome in considering curcumin for further drug development has been its relatively low bioavailability (Mock, M. et al. 2001). Despite this, studies by Zhang at al. show that curcumin and CMC2.24 bind fairly strongly to bovine serum albumin (Zhang, Y.; Golub L. M. et al. 2012), and when considering normal plasma concentrations of serum albumin, this should provide sufficient capacity to carry high enough concentrations of curcumin or CMC2.24 through the blood, increasing the half-time of their decomposition from mere minutes to tens of hours or days. In this same study, curcumin and CMC2.24 administered by oral gavage to rats expressing pathologically excessive levels of MMPs showed no evidence of toxicity, even in doses as high as 500 mg/kg of body weight (Zhang, Y. et al. 2012). Through chemical modification, it has now proven possible to synthesize derivatives of curcumin that have improved solubility, stability, and potential bioavailability, while still retaining or improving upon the inhibitory potency and negligible toxicity of the parent compound.

Four such CMCs, synthesized as reported previously (Zhang, Y. et al. 2012), were examined for inhibition of LF in vitro, in addition to curcumin itself (FIG. 1). Some of these CMCs have been found to have inhibitory potencies greater than or equal to curcumin itself against several of the matrix metalloproteinases, and this provided sufficient evidence that they may also exhibit comparable inhibition against LF. One of these CMCs in particular, CMC2.24, has shown exceptional promise in other systems, and is thus given prominence in this and other papers.

CMC2.24 shows improved solubility and even less toxicity in cell and tissue culture, as well as in in vivo studies, when compared to the parent compound (Zhang, Y. et al. 2012). The modifications to curcumin in synthesizing CMC2.24 include subtraction of the methoxy groups from the 3′ positions of curcumin's flanking aromatic rings, as well as the addition of a phenyl group, which is connected to the center of the molecule via a peptide bond. This modification provides CMC2.24 with an additional carbonyl capable of participating in keto-enol tautomerization, as well as several additional resonance structures, and a third hydrophobic region at its periphery. Studies by Zhang et al. show that CMC2.24 is nearly 10-fold more acidic than curcumin itself (Zhang, Y.; Golub L. M. et al. 2012), and exists largely as an enolate rather than an enol at physiological pH, which is likely a consequence of the additional electron-withdrawing group. This difference also seems responsible for CMC2.24's greater solubility, and superior zinc-binding ability (Zhang, Y.; Golub L. M. et al. 2012).

Anthrax

Lethal factor is just one component of the complex with protective antigen known as lethal toxin, and further assays of the curcuminoids' ability to inhibit this holotoxin are investigated to determine whether the compounds retain their inhibitory potency in cell-based assays that more closely model events in vivo. Considering the mixture of inhibitory mechanisms at work in this system, it is possible that the curcuminoids' capacity to inhibit LF by binding and inducing a conformational change in the enzyme could also have an effect on its PA binding domain, thus inhibiting not just the proteinase component of the toxin, but also the fundamental sequence of events that result in entry of LF into cells. The same end result could also be achieved if, along with their effects on the proteolytic activity of LF, the curcuminoids also bind to PA at its LF binding domain. Either of these scenarios could sufficiently compromise the in vivo activity of LeTx to neutralize its contribution to the pathology of anthrax. If the curcuminoids were indeed capable of binding to PA, they could likely also serve as inhibitors of edema toxin, and provide an all-encompassing means of combating the effects of anthrax infection.

It is not entirely necessary for a LF inhibitor to be able to enter the cell on its own. Rather, it can suffice for an inhibitor to bind LF in the extracellular space and travel with it into the cell, or to prevent it from binding PA at all. In just one example, such mechanisms may apply to some hydroxamate inhibitors of LF (Ammon, H. P. T. et al. 1991). Cellular uptake of curcumin has been quantitated and its localization throughout the cell demonstrated, particularly in the plasma membrane (Kunwar et al. 2008). Given the improved solubility and bioavailability of CMC2.24 (Zhang et al. 2012), as well as the overall lipophilic nature of the parent compound (Kunwar et al. 2008), evidence would suggest that cellular uptake of the CMCs should be comparable to the parent compounds. Moreover, considering the mixture of inhibitory mechanisms at work in this system, it is entirely possible that the curcuminoids' capacity to inhibit LF by binding and inducing a conformational change in the enzyme could also have an effect on its PA binding domain, thus inhibiting not just the proteinase component of the toxin, but also the fundamental sequence of events that result in entry of LF into cells.

Though curcumin appears ostensibly to be a more potent inhibitor of lethal factor in these in vitro studies, CMC2.24's superior stability, solubility, and ability to bind serum show that it is also therapeutic agent for treating anthrax (Zhang, Y.; GOlub, L. M. et al. 2012). If it can be shown that the curcuminoids are capable of in vitro inhibition of not just LF but LeTx as well, then experiments with cell lines should next be conducted to determine cell permeability to each individual candidate CMC. On the basis of such cell-based studies, tests of the compound in animal models can then be contemplated and improved methods for delivering the compound can be devised that are specific to the site and type of infection, such as aerosols or topical or oral formulations for inhaled, cutaneous, and gastrointestinal infections, respectively.

The data thus far points to an inhibitory mechanism other than competitive inhibition, a conclusion consistent with other reports of inhibition of LF by other polyphenolic inhibitors of LF, including the catechins (Tonello, F. et al. 2009; Dell'Aica, I. et al. 2004; Gaddis, B. D. et al. 2008; Numa, M. M. D. et al. 2005). The zinc-binding capacity of the curcuminoids was part of the basis for our initial motivation to explore their efficacy against LF, and though our data does not indicate that they inhibit the enzyme by binding the zinc atom in its active site, studies by Kochi et al. have shown evidence of up to three zinc atoms present in LF (Kochi, S. K. et al. 1994), any or all of which may be involved in the binding of the curcuminoids to LF at one or more allosteric sites. It also appears that the presence of hydroxyl groups at the 4′ positions of these molecules may play an important role in the binding of these compounds to LF, as indicated by the inferior inhibitory potency of CMC2.2 and CMC2.4, which lack them.

### SUMMARY

The data contained herein provides strong evidence that curcumin and CMC2.24 act to inhibit lethal factor by both decreasing its catalytic capacity and increasing its substrate affinity. The apparent increase in substrate affinity is mutually exclusive with a mechanism of competitive inhibition, and thus rules out simple occupancy of the substrate binding site by the inhibitor as an explanation for decreased enzyme activity. This also requires that curcumin and CMC2.24 bind to LF at an allosteric site, and therefore may function as uncompetitive or non-competitive inhibitors, though failure to fit the standard model of either of these mechanisms provides evidence for a mixture of inhibitory mechanisms. Two scenarios are possible to explain these observations. One possibility is that a single curcumin or CMC2.24 molecule may be capable of binding to an allosteric site on LF, and may affect both affinity for substrate and catalytic activity by inducing a conformational change in the enzyme. A second possibility is that there is more than one binding site for curcumin or CMC2.24 molecules on LF, one of which may affect catalysis and the other of which may affect substrate binding; when both sites are occupied, they may both contribute to the conformational changes necessary to affect substrate binding and catalysis. The presence of zinc atoms at these allosteric sites could provide a chemical basis for inhibitor binding. The data thus far points to an inhibitory mechanism other than competitive inhibition, a conclusion consistent with other reports of inhibition of LF by other inhibitors of LF with aromatic rings bearing hydroxyl or oxo groups, such as the gallocatechins (Tonello, F. et al. 2009; Dell'Aica, I. at al. 2004; Gaddis, B. D. at al. 2008; Numa M. M. at al. 2005).

The use of compounds such as curcumin in general, are important in the context of a bioterror attack: if developed further, curcumin and related compounds could potentially be used as post-exposure prophylactic agents, taken immediately after a bioterror attack is initiated, to prevent infection. Furthermore, if the additional metalloproteinase activities of B. anthracis as described by Popov et al. are also inhibited by curcuminoids, the compounds' broad specificity could be lifesaving (Popov, S. G. et al. 2005).

Several reports have pointed out that the specific cause of death from anthrax is still poorly understood. Although the role of lethal factor in pathogenesis has been defined, and the central role of the MAP Kinase cascade has been established in many cellular functions, inhibition of the MEKs alone does not seem sufficient to account for the systemic organ failure and tissue necrosis that is often seen in animals that have been infected with B. anthracis, or the rapid morbidity seen in Fischer Rats, which typically die within an hour from the time of exposure (Tonello, F. et al. 2009; Ezzel, J. W. et al. 1984). Regardless, previous studies have demonstrated that compounds that inhibit LF in vitro have prevented the death of Fischer Rats after intravenous injection of normally lethal amounts of LeTx (Dell'Aica, I. et al. 2004), suggesting that inhibition of enzyme activity in vitro may be truly correlated with prevention of morbidity and mortality in vivo. Realistically, treating anthrax effectively may ultimately require some form of combination therapy: antibiotics to combat the bacteria that are the ultimate source of pathogenesis, and small molecule inhibitors specifically directed against the actions of the toxic components produced by the bacteria. The promise of these compounds as therapeutic agents for the treatment of anthrax infection should therefore not be underestimated.

### REFERENCES

Abrami L.; Liu S.; Cosson P.; Leppla S. H.; van der Goot F. G. Anthrax toxin triggers endocytosis of its receptor via a lipid raft-mediated clathrin-dependent process. Journal of Cell Biology, 2003, 160(3), 321-328.

Ammon H. P. T.; Wahl M. A. Pharmacology of Curcuma longa. Planta Med, 1991, 57, 1-7.

Brittingham K. C.; Ruthel G.; Panchal R. G.; Fuller C. L.; Ribot W. J.; Hoover T. A.; Young H. A.; Anderson A. O.; Bavari S. Dendritic Cells Endocytose Bacillus anthracis Spores: Implications for Anthrax Pathogenesis. The Journal of Immunology, 2005, 174, 5545-5552.

Brossier F, Mock M. Toxins of Bacillus anthracis. Toxicon: official journal of the International Society on Toxinology. 2001 November; 39(11):1747-55. PubMed PMID: 11595637.

Dell'Aica I.; Donà M.; Tonello F.; Piris A.; Mock M.; Montecucco C.; Garbisa, S. Potent inhibitors of anthrax lethal factor from green tea. EMBO reports, 2004, 5(4), 418-422.

Dixon T. C.; Meselson M.; Guillemin J.; Hanna P. C. Anthrax. New England Journal of Medicine, 1999, 341(11), 815-826.

Ezzel J. W.; Ivins B. E.; Leppla S. H. Immunoelectrophoretic analysis, toxicity, and kinetics of in vitro production of the protective antigen and lethal factor components of Bacillus anthracis toxin. Infection and Immunity, 1984, 45(3), 761-767.

Ezzell J W, Jr., Abshire T G. Serum protease cleavage of Bacillus anthracis protective antigen. Journal of general microbiology. 1992 March; 138(3):543-9.

Gaddis B. D.; Rubert Pérez C. M.; Chmielewski J. Inhibitors of anthrax lethal factor based upon N-oleoyldopamine. Bioorganic & Medicinal Chemistry Letters, 2008, 18, 2467-2470.

Gupta S. C.; Prasad S.; Kim J. H.; Patchva S.; Webb L. J.; Priyadarsini I. K.; Aggarwal B. B. Multitargeting by curcumin as revealed by molecular interaction studies. Natural Products Reports, 2011, 28, 1937-1955.

Hellmich K A, Levinsohn J L, Fattah R, Newman Z L, Maier N, Sastalla I, et al. Anthrax lethal factor cleaves mouse nlrp1b in both toxin-sensitive and toxin-resistant macrophages. PloS one. 2012; 7(11):e49741.

Kocer S. S.; Walker S. G.; Zerler B.; Golub L. M.; Simon S. R. Metalloproteinase Inhibitors, Nonantimicrobial Chemically Modified Tetracyclines, and Ilomastat Block Bacillus anthracis Lethal Factor Activity in Viable Cells. Infection and Immunity, 2005, 73(11), 7548-7557.

Kochi S. K.; Schiavo G.; Mock M.; Montecucco C. Zinc content of the Bacillus anthracis lethal factor. FEMS Microbiology Letters, 1994, 124(3), 343-348.

Kunwar A, Barik A, Mishra B, Rathinasamy K, Pandey R, Priyadarsini K I. Quantitative cellular uptake, localization and cytotoxicity of curcumin in normal and tumor cells. Biochimica et biophysica acta. 2008 April; 1780(4):673-9.

Levinsohn J L, Newman Z L, Hellmich K A, Fattah R, Getz M A, Liu S, et al. Anthrax lethal factor cleavage of Nlrp1 is required for activation of the inflammasome. PLoS pathogens. 2012; 8(3):e1002638.

Mastrolorenzo A, Supuran C T. Botulinus Toxin, Tetanus Toxin, and Anthrax Lethal Factor Inhibitors. Drug Design of Zinc-Enzyme Inhibitors: John Wiley & Sons, Inc.; 2009. p. 705-720.

Mock M.; Fouet A. Anthrax. Annual Review of Microbiology, 2001, 55, 647-671.

Mock M.; Mignot T. Anthrax toxins and the host: a story of intimacy. Cellular Microbiology, 2003, 5(1), 15-23.

Numa M. M. D.; Lee L. V.; Hsu C.; Bower K. E.; Wong C. Identification of Novel Anthrax Lethal Factor Inhibitors Generated by Combinatorial Pictet-Spengler Reaction Followed by Screening in situ. ChemBioChem, 2005, 6, 1002-1006.

Pabon H. H. J. Synthesis of Curcumin and Related compounds. Rec. Trav. Chim. 83, 379-386, (1964).

Pannifer A. D.; Wong T. Y.; Schwarzenbacher R.; Renatus M.; Petosa C.; Bienkowska J.; Lacy D. B.; Collier R. J.; Park S.; Leppla S. H.; Hanna P.; Liddington R. C. Crystal structure of the anthrax lethal factor. Nature, 2001, 414, 229-233.

Popov S. G.; Popova T. G.; Hopkins S.; Weinstein R. S.; MacAfee R.; Fryxell K. J.; Chandhoke V.; Bailey C.; Alibek K. Effective antiprotease-antibiotic treatment of experimental anthrax. BMC Infectious Diseases, 2005, 5, 25.

Rosenberger C. M.; Finlay B. B.; Phagocyte Sabotage: Disruption of Macrophage Signaling By Bacterial Pathogens. Nature, 2003, 4, 385-396.

Rosenberger C M, Finlay B B. Phagocyte sabotage: disruption of macrophage signaling by bacterial pathogens. Nature reviews Molecular cell biology. 2003 May; 4(5):385-96.

Spyroulias G. A.; Galanis A. S.; Pairas G.; Manessi-Zoupa E.; Cordopatis P. Structural Features of Angiotensin-I Converting Enzyme Catalytic Sites: Conformational Studies in Solution, Homology Models and Comparison with Other Zinc Metallopeptidases. Current Topics in Medicinal Chemistry, 2004, 4(4), 403-429.

Supuran C T, Scozzafava A, Clare B W. Bacterial protease inhibitors. Medicinal research reviews. 2002 July; 22(4):329-72.

Tonello F.; Montecucco C; The anthrax lethal factor and its MAPK kinase-specific metalloprotease activity. Molecular Aspects of Medicine, 2009, 30, 431-438.

Zhang Y.; Golub L. M.; Johnson F.; Wishnia A. pKa, Zinc- and Serum Albumin-Binding of Curcumin and Two Novel Biologically-Active, Chemically-Modified Curcumins. Current Medicinal Chemistry, 2012, 19(25), 4367-4375.

Zhang Y., Gu Y., Lee H. M., Hambardjieva E., Vrankova K., Golub L. M., Johnson F. Design, Synthesis, and Biological Activity of New Polyenolic Inhibitors of Matrix Metalloproteinases: A Focus on Chemically-Modified Curcumins. Current Medicinal Chemistry, 2012, 19(25), 4348-4358.

1. A method of inhibiting the binding of anthrax lethal factor with protective antigen comprising contacting the anthrax lethal factor with a compound having the structure: 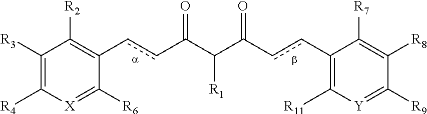
wherein
bond α and β are each, independently, present or absent;
X is CR₅ or N; Y is CR₁₀ or N;
R₁ is H, CF₃, halogen, —NO₂, —OCF₃, —OR₁₂, —NHCOR₁₂, —CONR₁₂R₁₃, —CSNR₁₂R₁₃, —C(═NH)NR₁₂R₁₃—SR₁₂, —SO₂R₁₃, —COR₁₄, —CSR₁₄, —C(═NR₁₂)R₁₄, —C(═NR₁₂)NR₁₃R₁₄, —SOR₁₂, —SONR₁₂R₁₃, —SO₂NR₁₂R₁₃, —P(O)R₁₂, —PH(═O)OR₁₂—P(═O)(OR₁₂)(OR₁₃), or —P(OR₁₂)(OR₁₃),
wherein R₁₂ and R₁₃ are each, independently, H, C₁₋₁₀ alkyl, C₂₋₁₀ alkenyl, C₂₋₁₀ alkynyl, aryl, heteroaryl, or heterocyclyl;
R₁₄ is C₂₋₁₀ alkyl, C₂₋₁₀ alkenyl, C₂₋₁₀ alkynyl, heteroaryl, heterocyclyl, methoxy, —OR₁₅, —NR₁₆R₁₇, or 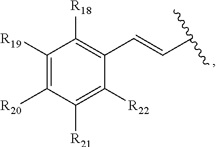
wherein R₁₅ is H, C₃₋₁₀ alkyl, C₂₋₁₀ alkenyl, C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
R₁₈, R₁₉, R₂₁, and R₂₂ are each independently H, halogen, —NO₂, —CN, —NR₂₃R₂₄, —SR₂₃, —SO₂R₂₃, —CO₂R₂₃, —OR₂₅, CF₃, —SOR₂₃, —POR₂₃, —C(═S)R₂₃, —C(═NH)R₂₃, —C(═N)R₂₃, —P(═O)(OR₂₃)(OR₂₄), —P(OR₂₃)(OR₂₄), —C(═S)R₂₃, C₁₋₁₀ alkyl, C₂₋₁₀ alkenyl, C₂₋₁₀ alkynyl, aryl, heteroaryl; or heterocyclyl;
wherein R₂₃, R₂₄, and R₂₅ are each, independently, H, C₁₋₁₀ alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl;
R₂, R₃, R₄, R₅, R₆, R₇, R₈, R₉, R₁₀, and R₁₁ are each independently, H, halogen, —NO₂, —CN, —NR₂₈R₂₉, —NHR₂₈R₂₉⁺, —SR₂₈, —SO₂R₂₈, —OR₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and
wherein when R₁ is H, then R₃, R₄, R₅, R₈, R₉, or R₁₀, is halogen, —NO₂, —CN, —NR₂₈R₂₉, —NHR₂₈R₂₉⁺, —SR₂₈, —SO₂R₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and
wherein each occurrence of alkyl, alkenyl, or alkynyl is branched or unbranched, unsubstituted or substituted;
or a pharmaceutically acceptable salt thereof.

2. The method of claim 1, wherein the compound inhibits anthrax lethal factor or anthrax lethal factor protease activity.

3. The method of claim 1, wherein the compound is a non-competitive inhibitor or uncompetitive inhibitor of anthrax lethal factor.

4. (canceled)

5. A method of treating a subject infected with Bacillus anthracis comprising administering to the subject a compound having the structure: 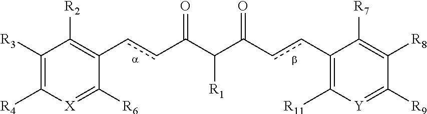
wherein
bond α and β are each, independently, present or absent;
X is CR₅ or N; Y is CR₁₀ or N;
R₁ is H, CF₃, halogen, —NO₂, —OCF₃, —OR₁₂, —NHCOR₁₂, —CONR₁₂R₁₃, —CSNR₁₂R₁₃, —C(═NH)NR₁₂R₁₃—SR₁₂, —SO₂R₁₃, —COR₁₄, —CSR₁₄, —C(═NR₁₂)R₁₄, —C(═NR₁₂)NR₁₃R₁₄, —SOR₁₂, —SONR₁₂R₁₃, —SO₂NR₁₂R₁₃, —P(O)R₁₂, —PH(═O)OR₁₂—P(═O)(OR₁₂)(OR₁₃), or —P(OR₁₂)(OR₁₃),
wherein R₁₂ and R₁₃ are each, independently, H, C₁₋₁₀ alkyl, C₂₋₁₀ alkenyl, C₂₋₁₀ alkynyl, aryl, heteroaryl, or heterocyclyl;
R₁₄ is C₂₋₁₀ alkyl, C₂₋₁₀ alkenyl, C₂₋₁₀ alkynyl, heteroaryl, heterocyclyl, methoxy, —OR₁₅, —NR₁₆R₁₇, or 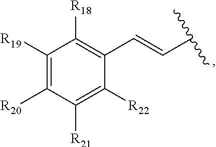
wherein R₁₅ is H, C₃₋₁₀ alkyl, C₂₋₁₀ alkenyl, C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
R₁₈, R₁₉, R₂₁, and R₂₂ are each independently H, halogen, —NO₂, —CN, —NR₂₃R₂₄, —SR₂₃, —SO₂R₂₃, —CO₂R₂₃, —OR₂₅, CF₃, —SOR₂₃, —POR₂₃, —C(═S)R₂₃, —C(═NH)R₂₃, —C(═N)R₂₃, —P(═O)(OR₂₃)(OR₂₄), —P(OR₂₃)(OR₂₄), —C(═S)R₂₃, C₁₋₁₀ alkyl, C₂₋₁₀ alkenyl, C₂₋₁₀ alkynyl, aryl, heteroaryl, or heterocyclyl;
wherein R₂₃, R₂₄, and R₂₅ are each, independently, H, C₁₋₁₀ alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl;
R₂, R₃, R₄, R₅, R₆, R₇, R₈, R₉, R₁₀, and R₁₁ are each independently, H, halogen, —NO₂, —CN, —NR₂₈R₂₉, —NHR₂₈R₂₉⁺, —SR₂₈, —SO₂R₂₈, —OR₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and
wherein when R₁ is H, then R₃, R₄, R₅, R₈, R₉, or R₁₀, is halogen, —NO₂, —CN, —NR₂₈R₂₉, —NHR₂₈R₂₉⁺, —SR₂₈, —SO₂R₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, C₂₋₁₀ alkynyl, or —C(═O)-heterocyclyl; and
wherein each occurrence of alkyl, alkenyl, or alkynyl is branched or unbranched, unsubstituted or substituted;
or a pharmaceutically acceptable salt thereof.

6. The method of claim 5, wherein the compound inhibits anthrax lethal factor or anthrax lethal factor protease activity in the subject infected with Bacillus anthracis.

7. The method of claim 5, wherein the compound inhibits the binding of anthrax lethal factor with protective antigen in the subject infected with Bacillus anthracis.

8. The method of claim 5, wherein the compound inhibits the binding of anthrax edema factor with protective antigen in the subject infected with Bacillus anthracis.

9. The method of claim 7, wherein the compound inhibits the formation of anthrax lethal toxin in the subject infected with Bacillus anthracis.

10. The method of claim 8, wherein the compound inhibits the formation of anthrax edema toxin in the subject infected with Bacillus anthracis.

11. The method of claim 5, further comprising administering an antibiotic to the subject infected with Bacillus anthracis.

12. The method of claim 1, wherein the compound has the structure: 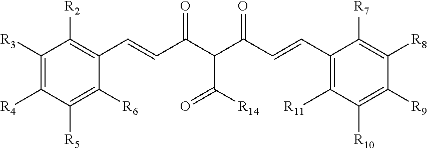
wherein R₁₄ is C₂₋₁₀ alkyl, C₂₋₁₀ alkenyl, C₂₋₁₀ alkynyl, heteroaryl, heterocyclyl, methoxy, —OR₁₅, —NR₁₆R₁₇, or 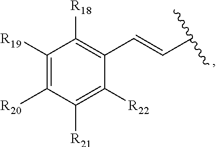
wherein R₁₅ is H, C₃₋₁₀ alkyl, C₂₋₁₀ alkenyl, C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
R₁₈, R₁₉, R₂₁, and R₂₂ are each independently H, halogen, —NO₂, —CN, —NR₂₃R₂₄, —SR₂₃, —SO₂R₂₃, —CO₂R₂₃, —OR₂₅, CF₃, C₁₋₂₀ alkyl, C₂₋₁₀ alkenyl, C₂₋₁₀ alkynyl, aryl, heteroaryl, or heterocyclyl;
wherein R₂₃, R₂₄, and R₂₅ are each, independently, H, C₁₋₁₀ alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl;
R₂, R₃, R₄, R₅, R₆, R₇, R₈, R₉, R₁₀, and R₁₁ are each independently, H, halogen, —NO₂, —CN, —NR₂₈R₂₉, —SR₂₈, —SO₂R₂₈, —OR₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, or C₂₋₁₀ alkynyl; and
wherein each occurrence of alkyl, alkenyl, or alkynyl is branched or unbranched, unsubstituted or substituted; and
or a salt thereof.

13. The method of claim 12, wherein at least one of R₂, R₃, R₄, R₅, and R₆ and at least one of R₇, R₈, R₉, R₁₀, and R₁₁, are each, independently, —OR₂₈.

14. The method of claim 12,
wherein
R₁₄ is methoxy, —OR₁₅ or —NR₁₆R₁₇;
R₁₅ is H, C₃₋₁₀ alkyl, C₂₋₁₀ alkenyl, or C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
or a salt thereof.

15-18. (canceled)

19. The method of claim 12, wherein the compound has the structure 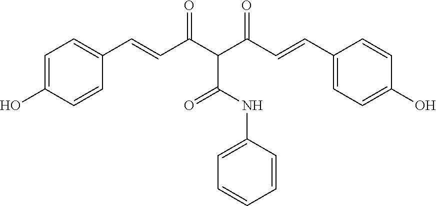 [SMILES: O=C(/C=C/c1ccc(O)cc1)C(C(=O)/C=C/c1ccc(O)cc1)C(=O)Nc1ccccc1]
or a pharmaceutically acceptable salt thereof.

20. The method of claim 12, wherein the compound has the structure 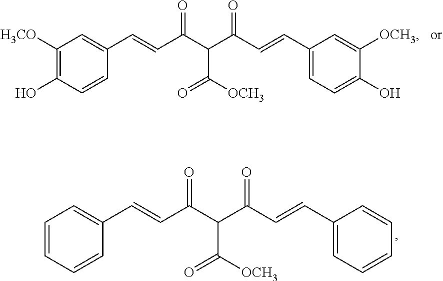 [SMILES: COC(=O)C(C(=O)/C=C/c1ccc(O)c(OC)c1)C(=O)/C=C/c1ccc(O)c(OC)c1 ; COC(=O)C(C(=O)/C=C/c1ccccc1)C(=O)/C=C/c1ccccc1]
or a pharmaceutically acceptable salt thereof.

21. (canceled)

22. (canceled)

23. The method of claim 5, wherein the compound has the structure: 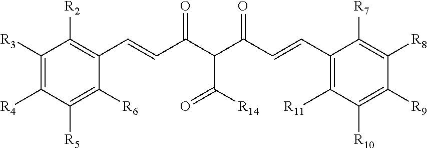
wherein R₁₄ is C₂₋₁₀ alkyl, C₂₋₁₀ alkenyl, C₂₋₁₀ alkynyl, heteroaryl, heterocyclyl, methoxy, —OR₁₅, —NR₁₆R₁₇, or 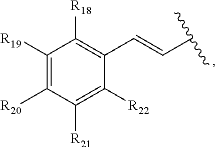
wherein R₁₅ is H, C₃₋₁₀ alkyl, C₂₋₁₀ alkenyl, C₂₋₁₀ alkynyl;
R₁₆ and R₁₇ are each independently, H, C₁₋₁₀ alkyl, C₂₋₁₀ alkenyl, C₂₋₁₀ alkynyl, aryl, heteroaryl, or heterocyclyl;
R₁₈, R₁₉, R₂₁, and R₂₂ are each independently H, halogen, —NO₂, —CN, —NR₂₃R₂₄, —SR₂₃, —SO₂R₂₃, —CO₂R₂₃, —OR₂₅, CF₃, C₁₋₁₀ alkyl, C₂₋₁₀ alkenyl, C₂₋₁₀ alkynyl, aryl, heteroaryl, or heterocyclyl;
wherein R₂₃, R₂₄, and R₂₅ are each independently, H, C₁₋₁₀ alkyl, C₂₋₁₀ alkenyl, C₂₋₁₀ alkynyl, aryl, heteroaryl, or heterocyclyl;
R₂₀ is halogen, —NO₂, —CN, —NR₂₆R₂₇, CF₃, C₁₋₁₀ alkyl, C₂₋₁₀ alkenyl, C₂₋₁₀ alkynyl, aryl, heteroaryl, or heterocyclyl;
wherein R₂₆ and R₂₇ are each, independently, H, C₁₋₁₀ alkyl, C₂₋₁₀ alkenyl, C₂₋₁₀ alkynyl, aryl, heteroaryl, or heterocyclyl;
R₂, R₃, R₄, R₅, R₆, R₇, R₈, R₉, R₁₀, and R₁₁ are each' independently, H, halogen, —NO₂, —CN, —NR₂₈R₂₉, —SR₂₈, —SO₂R₂₈, —OR₂₈, —CO₂R₂₈, CF₃, C₁₋₁₀ alkyl, C₂₋₁₀ alkenyl, C₂₋₁₀ alkynyl, aryl, heteroaryl, or heterocyclyl;
wherein R₂₈ and R₂₉ are each, H, CF₃, C₁₋₁₀ alkyl, C₂₋₁₀ alkenyl, or C₂₋₁₀ alkynyl; and
wherein each occurrence of alkyl, alkenyl, or alkynyl is branched or unbranched, unsubstituted or substituted; and
or a salt thereof.

24. The method of claim 23, wherein at least one of R₂, R₃, R₄, R₅, and R₆ and at least one of R₇, R₈, R₉, R₁₀, and R₁₁, are each independently, —OR₂₈.

25. The, method of claim 23,
wherein
R₁₄ is methoxy, —OR₁₅ or —NR₁₆R₁₇;
R₁₅ is H, C₃₋₁₀ alkyl, C₂₋₁₀ alkenyl, or C₂₋₁₀ alkynyl;
R₁₆ and R₁₇ are each, independently, H, C₁₋₁₀ alkyl, C₂₋₁₀ alkenyl, C₂₋₁₀ alkynyl, aryl, heteroaryl, or heterocyclyl;
or a salt thereof.

26. The method of claim 23, wherein the compound has the structure 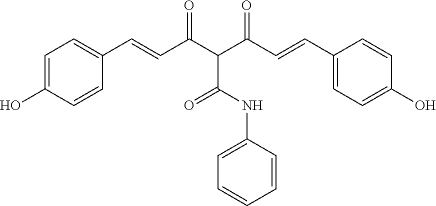 [SMILES: O=C(/C=C/c1ccc(O)cc1)C(C(=O)/C=C/c1ccc(O)cc1)C(=O)Nc1ccccc1]
or a pharmaceutically acceptable salt thereof.

27. The method of claim 23, wherein the compound has the structure 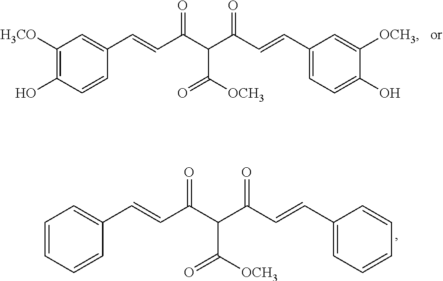 [SMILES: COC(=O)C(C(=O)/C=C/c1ccc(O)c(OC)c1)C(=O)/C=C/c1ccc(O)c(OC)c1 ; COC(=O)C(C(=O)/C=C/c1ccccc1)C(=O)/C=C/c1ccccc1]
or a pharmaceutically acceptable salt thereof.
